(19) United States
(12) Patent Application Publication
(10) Pub. No.: US 2007/0259774 A1
(43) Pub. Date: Nov. 8, 2007

(54) Enantioselective Phosphoramidite Compounds and Catalysts

(75) Inventors: John F. Hartwig, Champaign, IL (US); Andreas Leitner, Rainbach i. M. (AT); Chutian Shu, Danburg, CT (US)

Correspondence Address: COLEMAN SUDOL SAPONE, P.C., 714 COLORADO AVENUE, BRIDGE PORT, CT, 06605-1601, US

(73) Assignee: YALE UNIVERSITY, New Haven, CT (US)

(21) Appl. No.: 11/579,221

(22) PCT Filed: Apr. 29, 2005
(86) PCT No.: PCT/US05/15000

§ 371 Date: Feb. 14, 2007

Related U.S. Application Data
(60) Provisional application No. 60/566,969, filed on Apr. 29, 2004.

(51) Int. Cl. B01J 31/18; C07C 41/32; C07C 5/02; C07F 9/22

(52) U.S. Cl. 502/155; 568/12; 568/579; 585/277

(57) ABSTRACT

This invention relates to phosphoramidite compounds and catalyst complexes which can be used to provide enantioselective reactions including hydroamination reactions, etherification reactions and conjugate addition reactions and allylic substitution reactions, among others. In a first aspect, the present invention is directed to phosphoramidite and related compounds according to general structure (I), where Z is absent or is a group containing O, N or S, preferably O; R¹ and R² are independently an optionally substituted C₁₋₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminohiolate or a alcoholthiol group; R^{3′} and R³ are each independently H, an optionally substituted C₁-C₁₂ alkyl group or an optionally substituted (CH₂)ₙ-aromatic group with the proviso that R^{3′} and R³ are not both H, or together R^{3′} and R³ form an optionally substituted C₅-C₁₅ saturated or unsaturated carbocyclic ring; R⁴ is H, an optionally substituted C₁-C₁₂ alkyl group or an optionally substituted (CH₂)ₙ-aromatic group; R⁵ is absent, H, an optionally substituted C₁-C₁₂ alkyl group or an optionally substituted (CH₂)ₙ-aromatic or (CH₂)ₙ-heteroaromatic group; R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring; R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, heterocyclic ring or an aromatic or heteroaromatic ring, R⁵ is absent or is preferably H; R⁶ and R⁷ are preferably H or CH₃; and each n is independently 0, 1, 2, 3, 4, 5 or 6 and wherein at least one of the carbon atoms attached to the nitrogen of the phosphoramidite group is a chiral center.

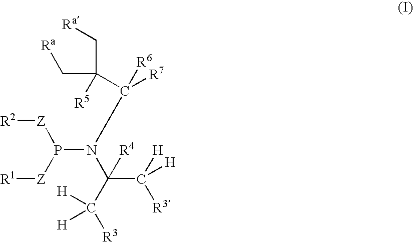

### RELATED APPLICATIONS

This application claims the benefit of priority of U.S. provisional application U.S. 60/566,969, filed Apr. 29, 2004, which is incorporated by reference in its entirely herein.

### GOVERNMENT SUPPORT

This invention was made in part with government support under grant number GM-55382 from the National Institutes of Health of the United States Department of Health and Human Services. Consequently, the U.S. Government has certain rights in the invention.

### FIELD OF THE INVENTION

This invention relates to phosphoramidite compounds and catalyst complexes which can be used to provide enantioselective reactions including hydroamination reactions, etherification reactions and conjugate addition reactions and allylic substitution reactions, among others.

### BACKGROUND OF THE INVENTION

Transition metal-catalyzed allylic substitution is a powerful tool for the controlled formation of carbon-carbon and carbon-heteroatom bonds (Godleski, S. A.; Trost, B. M., Fleming, I., Eds.; Pergamon Press: New York, 1991; Vol. 4, pp 585-661). Most enantioselective versions of these reactions with carbon nucleophiles have been reported with Pd (Jacobsen, E. N. et al., Comprehensive Asymmetric Catalysis I-III; Springer-Verlag: Berlin, Germany, 1999), but enantioselective allylic alkylation has also been reported with Mo (Trost, B. M.; Hachiya, I. J. Am. Chem. Soc. 1998, 120, 1104; Trost, B. M.; Hildbrand, S.; Dogra, K. J. Am. Chem. Soc. 1999, 121, 10416; Malkov, A. V.; Baxendale, I. R.; Dvorak, D.; Mansfield, D. J.; Kocovsky, P. joc 1999, 64, 2737), W (Lloyd-Jones, G. C.; Pfaltz, A. Angew. Chem., Int. Ed. 1995, 34, 462; Malkov, A. V.; Baxendale, I. R.; Dvorak, D.; Mansfield, D. J.; Kocovsky, P. joc 1999, 64, 2737), and, most recently, Ir catalysts (Takeuchi, R. Synlett 2002, 1954; Takeuchi, R.; Ue, N.; Tanabe, K.; Yamashita, K.; Shiga, N. J. Am. Chem. Soc. 2001, 123, 9525; Bartels, B.; Garcia-Yebra, C.; Rominger, F.; Helmchen, G. Eur. J. Inorg. Chem. 2002, 2569). However, despite the importance of optically active reaction steps and syntheses, relatively few enantioselective allylic aminations and etherifications by reactions of heteroatom nucleophiles have been described.

Allylic substitution of acyclic allylic electrophiles catalyzed by W, Mo, Ru, Ir, and Rh complexes often generate the chiral branched substitution products. A number of enantioselective amination reactions of symmetrical 1,3-diphenylallyl carbonates and unsymmetrical branched allylic acetates along with a few examples of palladium-catalyzed asymmetric amination reactions of a terminal allylic ester or carbonate have been reported (Hayashi, T. et al., J. Am. Chem. Soc. 1989, 111, 6301-6311; You, S. et al., J Am. Chem. Soc. 2001, 123, 7471; Hayashi, T. et al., Tetrahedron Lett. 1990, 31, 1743-1746; Johannsen, M.; Jørgensen, K. A. Chem. Rev. 1998, 98, 1689-1708). Takeuchi (Takeuchi, R.; et al., J. Am. Chem. Soc. 2001, 123, 9525-9534) and Evans (Evans, P. A.; et al., J. Am. Chem. Soc. 1999, 121, 6761-6762) have shown that iridium and rhodium complexes of achiral phosphites catalyze the formation of branched amines, in some cases with conservation of enantiomeric excess. Helmchen reported enantioselective alkylation of branched allylic acetates with modest levels of enantiomeric excess (ee) (Bartels, B.; Helmchen, G. Chem. Commun. 1999, 741-742) in the presence of an iridium-phosphoramidite catalyst. Analogous enantioselective aminations occurred with ee's below 15%. A general, enantioselective allylic amination from an achiral, terminal allylic electrophile has not been accomplished.

Aryl ethers are common subunits of biologically active molecules. Apart from their use as precursors for the Claisen rearrangement (Wipf, P.; Trost, B. M., Fleming, I., Paquette, L. A., Eds.; Pergamon press: Oxford, 1991; Vol. 5, pp 827-874; Larock, R. C. Comprehensive Organic Transformations: A Guide to Functional Group Preparations; VCH Publishers, Inc: New York, 1989), aryl allyl ethers have not been used extensively as building blocks for natural product synthesis because methods for their enantioselective construction are limited. Two reports of stereospecific allylic etherification of branched carbonates catalyzed by Ru (Trost, B. M.; Fraisse, P. L.; Ball, Z. T. Angew. Chem., Int. Ed. 2002, 41, 1059) and Rh (Evans, P. A.; Leahy, D. K. J. Am. Chem. Soc. 2000, 122, 5012; Evans, P. A.; Leahy, D. K. J. Am. Chem. Soc. 2002, 124, 7882) were reported recently, and a few enantioselective palladium-catalyzed examples have been reported (Trost, B. M.; Toste, F. D. J. Am. Chem. Soc. 1995, 121, 4545; Trost, B. M.; Toste, F. D. J. Am. Chem. Soc. 1998, 120, 815; Trost, B. M.; Tsui, H.-C.; Toste, F. D. J. Am. Chem. Soc. 2000, 122, 3534). Elegant applications of the palladium-catalyzed chemistry for the synthesis of natural products demonstrates the potential of these building blocks in organic synthesis (Trost, B. M.; Toste, F. D. J. Am. Chem. Soc. 1998, 120, 9074; Trost, B. M.; Toste, F. D. J Am. Chem. Soc. 2000, 122, 11262; Trost, B. M.; Thiel, O. R.; Tsui, H.-C. J. Am. Chem. Soc. 2002, 124, 11616; Trost, B. M.; Tang, W. J. Am. Chem. Soc. 2002, 124, 14542) Thus, new, more general, enantioselective methods for the construction of allylic ethers would be synthetically valuable.

International Patent Publication WO 04/024684 discloses enantioselective amination and etherification reactions using catalyst complexes of phorphoramidate compounds and a transition metal selected from the group consisting of iridium, molybdenum and tungsten, which were shown to be useful for allylic amination and etherification reactions.

International Patent Publication WO 02/04466 discloses catalysts for asymmetric transfer hydrogenation, including a transition metal selected from rhodium and ruthenium, and a phosphoramidite ligand. This publication also discloses processes for the asymmetric transfer hydrogenation of an olefinically unsaturated compound, ketone, imine or oxime derivative in the presence of a hydrogen donor and a catalyst, wherein the catalyst includes a transition metal selected from rhodium, ruthenium, and iridium, and a ligand.

International Patent Publication WO 01/23088 discloses catalysts for asymmetric transfer hydrogenation using a transition metal catalyst and a nitrogen-containing enantiomerically enriched ligand, as well as processes for the preparation of enantiomerically enriched compounds using such catalysts. According to the invention, the transition metal is iridium, ruthenium, rhodium or cobalt, and the enantiomerically enriched ligand contains sulfur in the form of a thioether or a sulfoxide.

Bartels et al., (Bartels, B.; Garcia-Yebra, C.; Rominger, F.; Helmchen, G. Eur. J. Inorg. Chem. 2002, 2569-2586) discloses Ir-catalysed allylic alkylations of enantiomerically enriched monosubstituted allylic acetates using P(OPh)₃ as ligand. Lithium N-tosylbenzylamide was identified as a suitable nucleophile for allylic aminations.

We conducted the first highly enantioselective aminations and etherifications of allylic carbonates with phosphoramidite ligand Li containing a binaphtholate unit and a bis-phenethylamino group. We later showed that the active catalyst in these reactions is generated by cyclometalation at one methyl group of the phenethylamino group. This cyclometallation breaks the C₂ symmetry of the ligand and generates a product with only C₁ symmetry. With the information that the active catalyst is generated by cyclometallation induced by a basic reagent, we increased the scope of the process to encompass more weakly basic nucleophiles, such as aromatic amines. To do so, we conducted reactions with catalytic amounts of an aliphatic amine to induce cyclometallation or conducted the catalytic process after activation of the precatalyst with a volatile aliphatic amine.

In principle, the information on the cyclometallation of the catalyst might also allow one to prepare new ligands with structures chosen more rationally than the original one. Moreover, the cyclometallated structure provides a platform for studying the origin of the effects of the different stereochemical elements of the ligand and the interconnections between these elements and on enantioselectivity. The cyclometallated catalyst contains one stereocenter remote from the metal, one stereocenter at a carbon, β to the metal, and an axial chirality at the binaphthyl unit. Different diastereomers of the ligand could be prepared in a straightforward manner to test the origins of enantioselectivity. The relative importance of the different stereochemical elements and the basis for the difference in activity between catalysts generated from different diastereomeric ligands was not clear from the initial studies of the enantioselective iridium-catalyzed process or even after identifying the structure of the cyclometalated species.

We reported in communication form that a ligand with a resolved binaphyl group, one phenethyl group and one achiral N-benzyl group distal to the metal generates a complex that catalyzes the allylation of cinnamyl carbonate with enantioselectivities higher than 90%. although, this catalyst was much less reactive than the original catalyst, it did demonstrate that the principle that the more distal stereocenter could be omitted while maintaining high selectivity.

### SUMMARY OF THE INVENTION

The present invention provides readily obtained and relatively inexpensive phosphoramidite compounds which can be used to form catalyst complexes with iridium, rhodium, ruthenium, nickel, palladium, platinum, copper or silver for enantioselective and regioselective hydrogenation or transfer hydrogenation reactions, for catalytic conjugate addition reactions and allylic substitution reactions, among others.

In a first aspect, the present invention is directed to phosphoramidite and related compounds (which term includes enantiomers and diastereomers, etc.) according to general structure: 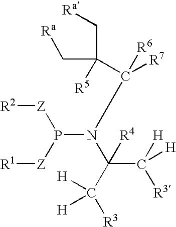
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic or (CH₂)ₙ-heteroaromatic group; R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, heterocyclic ring or an aromatic or heteroaromatic ring, R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center. The compound may be any diastereomer and any enantiomer containing these structural elements or a mixture of enantiomers and diastereomers.

In preferred aspects of the present invention, R¹ and R² are chosen to control the electronic properties of the central phosphorous group and can modulate the steric environment to help control the rate, regioselectivity and stereoselectivity of the reactions catalyzed by complexes of the phosphoramidite ligands according to the present invention. In other preferred aspects of the present invention, the carbon atom to which R³ or R^{3′} is attached, the carbon atom to which R⁴ is attached or the carbon to which R⁶ is attached is achiral (a non-stereocenter), a condition which makes the chemical synthesis of the ligand more facile, without compromising the activity and degree of chemical selectivity of catalyst complexes to which the phosphoramidite ligands have been bound.

In certain aspects of the present invention the group 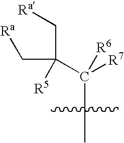
provides a substituted benzyl or naphthylmethyl group, with the substitution preferably being a methyl group on the methylene bridge connecting the aromatic group to the nitrogen. In such case, R⁵ is absent.

In other preferred aspects of the invention, the group 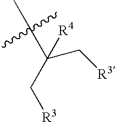
Provides an unsubstituted or substituted carbocyclic group wherein R^{3′} and R³ together with the carbon to which they are attached form a carbocyclic group and preferably R⁴ is H or a methyl group, more preferably H.

In other preferred aspects of the present invention, R¹ and R² are linked and form a biphenyl or binaphthyl group. In other preferred aspects of the invention, the chemical structure of the phosphoramidite is represented by the chemical structure: 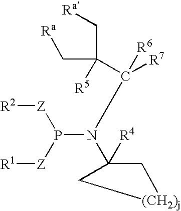
Where R¹, R², R⁴, R⁵, R⁶, R⁷, R^{a} and R^{a′} are the same as described above, and j is an integer from 2 to 12, preferably 3 to 9. R⁴ is preferably H or a methyl group, more preferably H, because of the ease of synthesis of such compounds.

Alternatively, in certain preferred aspects of the present invention, the carbon to which R⁴ is attached is a chiral center (stereocenter). Such compounds are represented by the two chemical structures below. 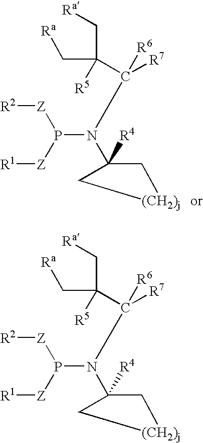
Where R¹, R², R⁴, R⁵, R^{a}, R^{a′}, R⁶ an R⁷ are the same as described above, and j is an integer from 2 to 12, preferably 3 to 9. R⁴ is preferably H or a methyl group, more preferably H, because of the ease of synthesis of such compounds.

The present compounds function as ligands in catalyst complexes according to the present invention.

In one aspect, the present invention is directed to a catalyst composition according to the structure:
MSXₙL
Where M is a transition metal which is preferably selected from the group consisting of iridium, tungsten, molydenum, rhodium, ruthenium, nickel, palladium, platinum, copper or silver, more preferably iridium, rhodium, ruthenium, nickel, palladium or platinum;
S is a coordinating ligand;
X is a counterion;
n is an integer from 0 to 6; and
L is a phosphoramidite ligand (which term includes enantiomers and diastereomers, etc.) as described hereinabove.
The above catalysts are useful for allylation reactions as otherwise described herein.
Alternative catalyst compositions for use in the present invention have the following structure:
M′SₘXₖL
Where M′ is a transition metal which is preferably selected from the group consisting of iridium, rhodium, ruthenium, copper or silver;
S is a coordinating ligand;
X is a counterion;
m is an integer from 0 to 6;
k is an integer from 0 to 6; and
L is a phosphoramidite ligand (which term includes enantiomers and diastereomers, etc.) as described hereinabove.
The above catalysts are useful for hydrogenation reactions (where M is preferably Ir, Ru or Rh) and conjugate addition reactions (where M is preferably Rh, Cu or Ag).

In another aspect, the present invention is directed to a method of making a catalyst as described, the catalyst comprising a metal complex of a phosphoramidite, comprising the step of combining a catalyst precursor MSXₙ and a phosphoramidite compound as otherwise disclosed herein in the presence of an optional base under conditions that form the catalyst MSXₙL. In preferred aspects of the present invention, M is a transition metal selected from the group consisting of iridium, rhodium, ruthenium, tungsten, molybdenum, nickel, palladium and platinum, more preferably iridium, rhodium, ruthenium, nickel, palladium or platinum. Catalysts according to this formula are preferably used in allylic substitution reactions according to the present invention.

In another aspect, the present invention is directed to a method of making a catalyst as described, the catalyst comprising a metal complex of a phosphoramidite, comprising the step of combining a catalyst precursor M′SₘXₖ and a phosphoramidite compound as otherwise disclosed herein in the presence of an optional base under conditions that form the catalyst M′SₘXₖL. In preferred aspects of the present invention, M′ is a transition metal selected from the group consisting of iridium, rhodium, ruthenium, copper or silver. Catalysts according to this formula are preferably used in hydrogenation reactions (where M is preferably Ir, Ru or Rh) and conjugate addition reactions (where M is preferably Rh, Cu or Ag) according to the present invention.

In yet another aspect, the present invention is directed to a method of preparing allylic amines enantioselectively, the method comprising the steps of reacting (a) an achiral or racemic allylic ester, allylic carbonate or allylic halide; and (b) a reactant containing an N—H bond or a salt thereof, in the presence of a solvent and a catalyst composition, the catalyst composition comprising (1) a catalyst precursor having the general structure MSXₙ wherein M is a transition metal selected from the group consisting of iridium, rhodium, ruthenium, tungsten, molybdenum, nickel, palladium and platinum, more preferably iridium, rhodium, ruthenium, nickel, palladium or platinum; S is a coordinating ligand; X is a counterion; and n is an integer from 0 to 5; and (2) a phosphoramidite ligand (which term includes enantiomers and diastereomers, etc.) according to general structure: 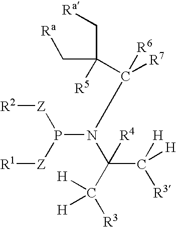
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, amnoalcohol, aminothiol or alcoholthiol group;
R^{3′} and R³ are each independently H, an optionally substituted C₁-C₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form an optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic or heterocyclic ring or an aromatic or heteroaromatic ring, R⁵ is absent or is preferably H; and R⁶ and R⁷ are preferably H or CH₃; and
each n is independently 0, 1, 2, 3, 4, 5 or 6 and wherein at least one of the carbon atoms attached to the nitrogen of the phosphoramidite group is a chiral center under conditions that enantioselectively form allylic amines.

In certain preferred aspects of the present invention related to a method of preparing allylic amines, the phosphoramidite compound (which term includes enantiomers and diastereomers, etc.) has the structure: 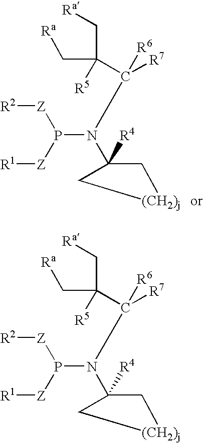
Where R¹, R², R⁴, R⁵, R^{a}, R^{a′}, R⁶ an R⁷ are the same as described above, and j is an integer from 2 to 12, preferably 3 to 9. R⁴ is preferably H or a methyl group, more preferably H, because of the ease of synthesis of such compounds.

In yet another aspect, the present invention is directed to a method of preparing allylic ethers enantioselectively, the method comprising the steps of reacting (a) an achiral or racemic allylic ester, allylic carbonate or allylic halide (b) a reactant containing an O—H bond, and (c) optionally, a base; the reacting step taking place in a solvent and in the presence of a catalyst composition, the catalyst composition comprising a transition metal selected from the group consisting of iridium, rhodium, ruthenium, tungsten, molybdenum, nickel, palladium and platinum, more preferably iridium, rhodium, ruthenium, nickel, palladium or platinum, the reacting step taking place under conditions that enantioselectively form allylic ethers.

In yet another aspect, the present invention is directed to a method of preparing allylic ethers enantioselectively, the method comprising the steps of reacting (a) an achiral or racemic allylic ester, allylic carbonate or allylic halide and (b) a reactant containing an O—H bond, or a salt thereof, the reacting step taking place in a solvent and in the presence of a catalyst composition, the catalyst composition comprising (1) a catalyst precursor having the general structure MSXₙ wherein M is a transition metal selected from the group consisting of iridium, rhodium, ruthenium, tungsten, molybdenum, nickel, palladium and platinum, more preferably iridium, rhodium, ruthenium, nickel, palladium or platinum; S is a coordinating ligand; X is a counterion; and n is an integer from 0 to 5; and (2) a phosphoramidite ligand L (which term includes enantiomers and diastereomers, etc.) having the structure: 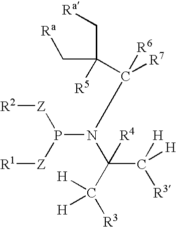
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, amnoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent or is H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring, or an aromatic or heteroaromatic ring,
R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center; under conditions that enantioselectively form allylic ethers.

In certain preferred aspects of the present invention related to a method of preparing allylic ethers, the phosphoramidite compound (which term includes enantiomers and diastereomers, etc.) has the structure: 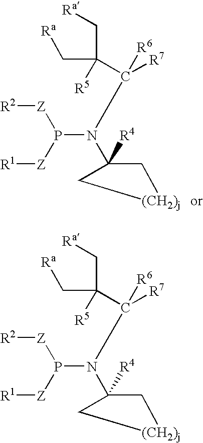
Where R¹, R², R⁴, R⁵, R^{a}, R^{a′}, R⁶ an R⁷ are the same as described above, and j is an integer from 2 to 12, preferably 3 to 9. R⁴ is preferably H or a methyl group, more preferably H, because of the ease of synthesis of such compounds.

In yet another aspect, the present invention is directed to a method of preparing products from formation of a carbon-carbon bond between (a) an achiral or racemic allylic ester, allylic carbonate or allylic halide and (b) an enolate derived from a cyanoacetate, β-cyanoketone, malonate, 1,3-diketone or other stabilized carbonanion, such as that from an azlactone or imine-protected α-aminoacid, the reacting step taking place in a solvent and in the presence of a catalyst composition, the catalyst composition comprising

(1) a catalyst precursor having the general structure MSXₙ wherein M is a transition metal selected from the group consisting of iridium, rhodium, ruthenium, tungsten, molybdenum, nickel, palladium and platinum, more preferably iridium, rhodium, ruthenium, nickel, palladium or platinum; S is a coordinating ligand; X is a counterion; and n is an integer from 0 to 6; and (2) a phosphoramidite ligand L (which term includes enantiomers and diastereomers, etc.) having the structure: 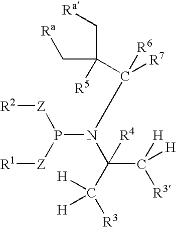
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, amnoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent or is H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;

R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring, or an aromatic or heteroaromatic ring, R⁵ is absent or is preferably H; R⁶ and R⁷ are preferably H or CH₃; and Each n is independently 0, 1, 2, 3, 4, 5 or 6 and wherein at least one of the carbon atoms attached to the nitrogen of the phosphoramidite group is a chiral center; under conditions that enantioselectively form allylic ethers.

In certain preferred aspects of the present invention related to a method of preparing allylic ethers, the phosphoramidite compound (which term includes enantiomers and diastereomers, etc.) has the structure: 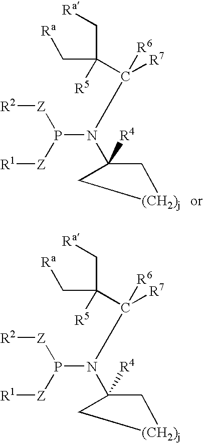
Where R¹, R², R⁴, R⁵, R^{a}, R_{a′}, R⁶ an R⁷ are the same as described above, and j is an integer from 2 to 12, preferably 3 to 9. R⁴ is preferably H or a methyl group, more preferably H, because of the ease of synthesis of such compounds.

A still further aspect of the present invention relates to an enantioselective hydrogenation reaction which occurs by reacting hydrogen with an olefin compound in the presence of a solvent, the reacting step taking place in a solvent and in the presence of a catalyst composition, the catalyst composition comprising (1) a catalyst precursor having the general structure M′SₘXₖ wherein M′ is a transition metal selected from the group consisting of iridium, rhodium or ruthenium; S is a coordinating ligand; X is a counterion; k is an integer from 0 to 6 and m is an integer from 0 to 6; and (2) a phosphoramidite ligand L (which term includes enantiomers and diastereomers, etc.) having the structure: 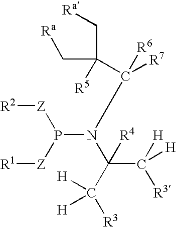
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, amnoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂),-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring or an aromatic or heteroaromatic ring, R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center under conditions which enantioselectively hydroaminate said olefin.

In certain preferred aspects of the present invention related to the enantioselective hydrogenation method, the phosphoramidite compound (which term includes enantiomers and diastereomers, etc.) has the structure: 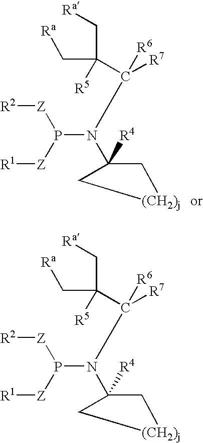
Where R¹, R², R⁴, R⁵, R^{a}, R^{a′}, R⁶ an R⁷ are the same as described above, and j is an integer from 2 to 12, preferably 3 to 9. R⁴ is preferably H or a methyl group, more preferably H, because of the ease of synthesis of such compounds.

A still further aspect of the present invention relates to a conjugate addition reaction which occurs by reacting a compound containing a nucleophilic group with an alpha, beta unsaturated carbonyl compound or nitroalkane, the reacting step taking place in a solvent and in the presence of a catalyst composition, the catalyst composition comprising (1) a catalyst precursor having the general structure M′SₘXₖ wherein M′ is a transition metal selected from the group consisting of rhodium, copper or silver; S is a coordinating ligand; X is a counterion; k is an integer from 0 to 6 and m is an integer from 0 to 6; and (2) a phosphoramidite ligand L (which term includes enantiomers and diastereomers, etc.) having the structure: 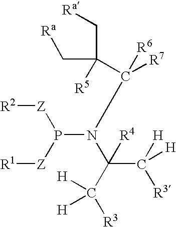
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring or an aromatic or heteroaromatic ring, R⁵ is absent or is preferably H, R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center under conditions which enantioselectively add the nucleophile to said conjugated compound.

In certain preferred aspects of the present invention related to a conjugate addition method, the phosphoramidite compound (which term includes enantiomers and diastereomers, etc.) has the structure: 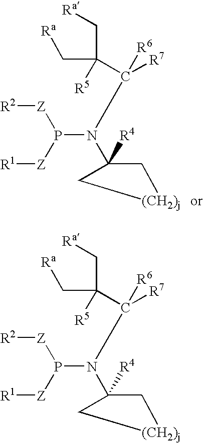
Where R¹, R², R⁴, R⁵, R^{a}, R^{a′}, R⁶ an R⁷ are the same as described above, and j is an integer from 2 to 12, preferably 3 to 9. R⁴ is preferably H or a methyl group, more preferably H, because of the ease of synthesis of such compounds.

These and other aspects will become apparent upon reading the following detailed description of the invention.

### BRIEF DESCRIPTION OF THE FIGURES

FIG. 1 is a diagrammatic representation of original phosphoramidite ligand L1 and activated cyclometallated catalyst 1.

FIG. 2 shows the stereochemical elements of the cyclometallated Ir(I)-complex generated with ligand L1.

FIG. 3 shows an ORTEP diagram of a trigonal bipyramidal Ir(I)-complex with PPh₃ as the monodentate phosphorus ligand.

FIG. 4 shows representative equation 1 the results of which are set forth in Table 1 in the experimental section of the present specification.

FIG. 5 shows a similar representative equation to that shows in FIG. 4, with a number of alternative catalysts used to effect the conjugate addition reaction.

FIG. 6 shows a representative equation of the effect of two catalysts as indicated on the reaction of aniline with methyl cinnamyl carbonate.

### DETAILED DESCRIPTION OF THE INVENTION

The following terms shall be used throughout the specification to describe the present invention, noting that terms which are not defined herein are given their plain meaning as understood by those of ordinary skill in the art.

The term “compound” is used to describe any chemical compound or ligand, especially a phosphoramidite ligand according to the present invention, which is used in the present invention and in context may refer to a purified or substantially pure compound or a less than pure compound or a compound complexed to a metal and coordinating ligand in a catalyst complex. In addition, compounds according to the present invention may refer to all optical (including enantiomeric and diastereomeric) isomers, regioisomers and/or stereoisomers within the context of use or synthesis and may include racemic mixtures and/or enantiomerically enriched compounds, individually or as mixtures. Purified and isolated compounds, especially phosphoramidite compounds according to the present invention are preferred in numerous embodiments.

The term “effective” is used to describe an amount of a compound or component which is used or included within the context of its use to provide an intended result. An effective amount may range quite broadly, within context, depending upon a number of factors, conditions, components and/or additives and the role that they play within the context of their use. One of ordinary skill will be able to determine an effective amount by routine experimentation, where such amount is not explicitly described.

The term “alkyl” is used herein to refer to a fully saturated, monovalent radical containing carbon and hydrogen, and which may be a straight chain, branched or cyclic. Examples of preferred alkyl groups include C₁-C₇ alkyl groups such as methyl, ethyl, n-butyl, n-pentyl, n-heptyl, isopropyl, 2-methylpropyl, cyclopropyl, cyclopropylmethyl, cyclobutyl, cyclopentyl, cyclopentylethyl and cyclohexyl.

The term “aromatic” or “aryl” refers to a substituted or unsubstituted monovalent aromatic radical having a single ring (e.g., phenyl) or multiple condensed rings (e.g., naphthyl, anthracene, phenanthrene, etc.). The term aromatic or aryl includes heteroaromatic radicals with nitrogen, oxygen or sulfur or a combination of these atoms in the ring system.

The term “substituted” is used to describe a substituent which in context may be incorporated onto a group of a phosphoramidite ligand or a reactant which is used in the present invention. Exemplary substituents which may be used in the present invention include hydrocarbon groups such as alkyl, alkenyl or aryl (aromatic) groups, which themselves may be unsubstituted or substituted, alkyl or aryl alkoxides (preferably, unsubstituted or substituted C₁-C₆ alkoxides or phenoxides), keto, ester or carboxylic acid groups, amines, which may be unsubstituted or substituted with substituents otherwise disclosed herein, halogens (F, Br, Cl, I), mono- and dialkylamido groups, mono- and diarylamido groups, amidates (preferably unsubstituted or substituted alkyl or aryl amidates), among numerous others. Exemplary preferred substituents include unsubstituted or substituted C₁-C₆ alkyl groups or aryl groups (especially halogenated alkyl groups), preferably phenyl groups, alkoxide groups, keto groups, keto esters, carboxyl groups or amino groups, which may be attached to a ligand or other substituent in context, through carbon, oxygen, nitrogen or sulfur atoms. Note that the term substituent subsumes or incorporates O, S, N, Si or P atoms within alkyl or alkylene chains and in particular, R¹ and R² substituents of the phosphoramidite compounds of the present invention.

The terms “compound with an N—H bond”, “N—H containing compound”, “amine-containing compound” and “amine” throughout the specification are used within context, and usually, but not exclusively, refer to a reactant which contains an N—H bond and participates in or is a final product produced by the allylic substitution reactions of the present invention. Representative reactants for use in the present invention include any compound with an N—H bond, for example, reactive amines, silylamines, hydrazones, amides, carbamates, sulfonamides, sulfoximines, imines, imides, heterocyclic, including heteroaryl compounds such as unsubstituted or substituted indoles, pyrroles, pyrazoles and imidazoles, among others, with reactive amines being preferred. Preferred reactive amines for use in the present invention include, for example, ammonia, substituted or unsubstituted aromatic or aliphatic primary or secondary amines, including aralkylamines, such as substituted or unsubstituted anilines, diphenylmethylamine, benzylamines, 4-methoxybenzylamine; primary alkylamines such as n-hexylamine and allyl amines; secondary cyclic amines such as pyrrolidine, piperidine, and morpholine; and acyclic secondary amines such as diethylamine; among numerous others.

The term “olefin” is used throughout the specification to describe certain reactants, which are used in hydrogenation reactions according to the present invention. An olefin is any compound with a carbon-carbon double bond which can participate in addition (hydroamination) reactions according to the present invention, and includes terminal and internal alkenes, vinylarenes, dienes, eneynes, and alpha-beta-unsaturated carbonyl compounds.

The term reagents or compounds “that contain an O—H bond” is directed to certain compounds which find use as etherification reactants according to the present invention include alkoxides, phenoxides, siloxides, carboxylates, phosphates, alcohols, phenols, silanols, carboxylic acids, hydroxylamines, phosphorus-containing acids, and salts thereof. Specific examples of useful reagents which are salts of compounds containing an O—H bond that may be employed in the etherification method of the invention include 2-MeC₆H₄OLi, 4-MeC₆H₄OLi, 4-MeOC₆H₄OLi, 3-MeOC₆H₄OLi, 3-PhC₆H₄OLi, 2-PhC₆H₄OLi, 3-Me₂NC₆H₄OLi, 3,4-(OCH₂O)C₆H₃OLi, 2,4-Me₂C₆H₃OLi, 2,4,6-Me₃C₆H₃OLi, 4-BrC₆H₄ONa, 4-ClC₆H₄ONa, 4-Br,3-MeC₆H₄ONa, 4-CF₃C₆H₄ONa, PhOLi, PhONa, as well as salts of these.

The terms “conjugated compound”, “diene compound” or “enone compound” refer to compounds which take part in conjugated addition reactions according to the present invention and are generally well known in the art. Specific examples of such compounds include compounds of the following structure: 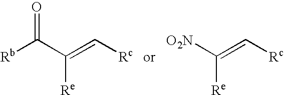
Where R^{b} is alkyl (preferably C₁-C₁₂, more preferably C₁-C₃ alkyl), aryl, alkoxy ((preferably C₁-C₁₂, more preferably C₁-C₃ alkoxy), phenoxy or amino; and
R^{c}is alkyl (preferably C₁-C₁₂, more preferably C₁-C₃ alkyl), vinyl (preferably C₂-C₁₂, more preferably C₂-C₄ vinyl), aryl or alkynyl (preferably C₂-C₁₂, more preferably C₂-C₄ alkynyl) group; and R^{e} is alkyl (preferably C₁-C₁₂, more preferably C₁-C₃ alkyl), vinyl (preferably C₂-C₁₂, more preferably C₂-C₄ vinyl), aryl, or alkynyl (preferably C₂-C₁₂, more preferably C₂-C₄ alkynyl), or any of R^{b}, R^{c} and Re together are connected to form a cyclic compound. These compounds can be acyclic or cyclic, as described. Examples of such compounds include cyclohexenone, 5,6-dihydro-2H-pyran-2-one, chalcone, beta-nitrostryene and the like.

The term “acid” refers to any acid, generally a protic or Lewis acid, preferably a protic acid, which includes acids having a pKa of less than about 2 (“strong acid”) or above about 2 (“weak acid”). Strong acids include, for example, trifluoromethylsulfonic acid (triflic acid), p-toluenesulfonic acid (HOTs), benzoic acid, trifluoroacetic acid and a number of other sulfonic acids and carboxylic acids. The strong acid may also be a Lewis acid, such as B(C₆F₅)₃, AgBF₄, AgOTF or other Lewis acids which are well known in the art. The term “weak acid” refers to an acid, preferably a protic acid, having a pKa of significantly greater than about 2 and includes many organic acids such as acetic acid, malic acid, mandelic acid, among numerous others.

The term “coordinating ligand” refers to ligands represented by the letter S in the catalyst precursor compositions (MSXₙ or M′SₘXⱼ or catalyst compositions (MSXₙL or M′SₘXⱼL) according to the present invention and include ethylene, maleic anhydride, 1,5-cyclooctadiene, cyclooctene, 1,3-butadiene, 2,3-dimethyl-1,3-butadiene, 2,5-norbornadiene, benzene, hexamethyl benzene, cymene, cumene, cyclopentadiene, pentamethylcyclopentadiene, 1,2-diaminoethane, (R,R)-1,2-cyclohexanediamine, (S,S)-1,2-diphenyl-1,2-diaminoethane, (S,S)-1,2-dicyclohexyl-1,2-diaminoethane, (S)-1,1′-bis-(p-methoxyphenyl)-1,2-propanediamine. Particularly useful coordinating ligands S are 1,5-cyclooctadiene (abbreviated as COD) and 2,5-norbornadiene. It will be understood that alternative enantiomers (R) and (S) of the above coordinating ligands may also be used. Further, as will be appreciated by one skilled in the art, combinations of the aforementioned coordinating ligands may also be implemented in the catalysts and methods of the present invention.

In the catalyst precursor, X is counterion which may be anionic or cationic. Useful counterions include, but are not limited to, Cl, Br, I, acetate, BF₄, PF₆, ClO₄, p-toluene sulfonate, benzene phosphonate, tetra-pentafluorophenylborate, Li, Na, K, Mg, Ca, ammonium, and alkyl-substituted ammonium. Like the coordinating ligands, combinations of counterions X may be implemented in the catalysts and methods of the present invention. The number of X counterions (n) in the MSX, catalyst precursor is sufficient to counterbalance the charge on the complex. Preferably, n can range from zero (0) to six (6). In a preferred embodiment, the catalyst precursor has the structure [(COD)IrCl]₂. The catalyst precursors may be made using published procedures known in the art, such as those described in Herde et al., Inorg. Synth. 15:18 (1974), herein incorporated by reference in its entirety.

Applicants have unexpectedly discovered that catalysts made from a transition metal-containing catalyst precursor and a phosphoramidite ligand as claimed are capable of catalyzing production of allylic amines and allylic esters, allylic alkylation products, the production of hydrogenation reaction products and the production of conjugate addition reaction products with high regio- and enantioselectivity. In the case of allylic amines and allylic ethers, the catalysts and methods of the present invention are useful in the preparation of materials containing a terminal olefin group. Such products may be used as precursors to generate other useful products, for example, 1,3-amino alcohols, 1,3-diamines, and various types of amino acids. Hydroamination reaction products and conjugate addition products as otherwise discussed herein are also advantageously produced by the catalysts and methods according to the present invention. Such products are useful in the chemical and pharmaceutical industries.

For the purposes of this application, the term “ester” includes compounds containing an oxygen bound to a carbon, phosphorus or sulfur that is bound to an additional oxygen through a multiple bond or a compound containing an oxygen bound to boron that is bound to two additional oxygen atoms.

As indicated above, the catalyst composition of the present invention comprises (1) a catalyst precursor having the general structure MSXₙ wherein M is a transition metal selected from the group consisting of iridium, tungsten and molydenum, rhodium, ruthenium, nickel, palladium, platinum, copper and silver; S is a coordinating ligand; X is a counterion; and n is an integer from 0 to 6; and (2) a phosphoramidite and related compounds (phosphoramidite ligand) according to general structure: 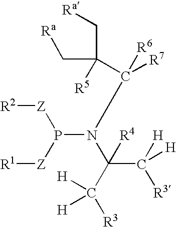
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a saturated or unsaturated carbocyclic ring or heterocyclic ring or an aromatic or heteroaromatic ring, R⁵ is absent or is preferably H, R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center.

In preferred aspects of the present invention, R¹ and R² are chosen to control the electronic properties of the central phosphorous group and can modulate the steric environment to help control the rate, regioselectivity and stereoselectivity of the reactions catalyzed by complexes of the phosphoramidite ligands according to the present invention. In other preferred aspects of the present invention, the carbon atom to which R³ or R^{3′} is attached, the carbon atom to which R⁴ is attached or the carbon to which R⁶ is achiral (a non-stereocenter), a condition which makes the chemical synthesis of the ligand more facile, without compromising the activity and degree of chemical selectivity of catalyst complexes to which the phosphoramidate ligands have been bound.

Each of these components is discussed in more detail below.

Transition metals useful in the catalyst precursor component of the invention include iridium, tungsten, molydenum, rhodium, ruthenium, nickel, palladium, platinum, copper or silver, more preferably iridium, rhodium, ruthenium, nickel, palladium or platinum, depending upon the reaction to be catalyzed, as described above.

In certain aspects, the catalyst precursor has a general structure MSXₙ, where S is a coordinating ligand for the transition metal M. Useful coordinating ligands S include, but are not limited to, ethylene, maleic anhydride, 1,5-cyclooctadiene, cyclooctene, 1,3-butadiene, 2,5-norbornadiene, benzene, hexamethyl benzene, cymene, cumene, cyclopentadiene, pentamethylcyclopentadiene, 1,2-diaminoethane, (R,R)-1,2 cyclohexanediamine, (S,S)-1,2-diphenyl-1,2-diaminoethane, (S,S)-1,2-dicyclohexyl-1,2-diaminoethane, and (S)-1,1′-bis-(p-methoxyphenyl)-1,2-propanediamine. Particularly useful coordinating ligands S are 1,5-cyclooctadiene (abbreviated as COD) and 2,5-norbornadiene. It will be understood that alternative enantiomers (R) and (S) of the above coordinating ligands may also be used. Further, as will be appreciated by one skilled in the art, combinations of the aforementioned coordinating ligands may also be implemented in the catalysts and methods of the present invention.

In the catalyst precursor described above, X is counterion which may be anionic or cationic. Useful counterions include, but are not limited to, Cl, Br, I, acetate, BF₄, PF₆, ClO₄, p-toluene sulfonate, benzene phosphonate, tetra-pentafluorophenylborate, Li, Na, K, Mg, Ca, ammonium, and alkyl-substituted ammonium. Like the coordinating ligands, combinations of counterions X may be implemented in the catalysts and methods of the present invention. The number of X counterions (n) in the MSXₙ catalyst precursor is sufficient to counterbalance the charge on the complex. Preferably, n can range from zero (0) to six (6). The catalyst precursors may be made using published procedures known in the art, such as those described in Herde et al., Inorg. Synth. 15:18 (1974), herein incorporated by reference in its entirety, or alternative references, well known in the art.

Alternative catalyst compositions include those of the general structure:
M′SₘXⱼL
Where M′ is a transition metal which is preferably selected from the group consisting of iridium, rhodium, ruthenium, copper or silver;
S is a coordinating ligand;
X is a counterion;
m is an integer from 0 to 6;
j is an integer from 0 to 6; and
L is a phosphoramidite ligand as described hereinabove.

The above catalysts M′SₘXⱼL are useful for hydrogenation reactions (where M is preferably Ir, Ru or Rh) and conjugate addition reactions (where M is preferably Cu or Ag). Coordinating ligands S are the same as those previously described.

The phosphoramidite portion of the catalyst composition of the invention may be any phosphoramidite as otherwise described herein, to provide and facilitate enantioselectivity and/or regioselectivity.

In certain preferred aspects, the phosphoramidite ligand has the structure: 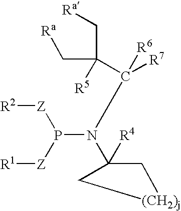
Or alternatively, the chemical structure is preferably 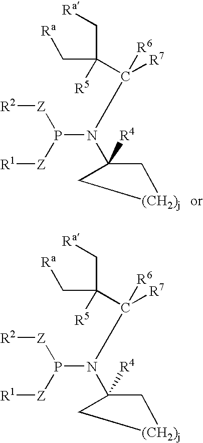
Where R¹, R², R⁴, R⁵, R⁶, R⁷, R^{a}, R^{a′} and j are as otherwise described hereinabove.

In the above structures, 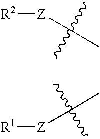
forms an O—Cₖ—O group and is preferably an aliphatic or aromatic diolate, as disclosed below. R⁴ is preferably H or CH₃, j is preferably 4-10, more preferably 5-9 (representing a cyclooctyl or cyclododecyl group), R^{a} and R^{a′} preferably form an optionally substituted aliphatic group, including a cycloalkyl group, an optionally substituted aromatic or heteroaromatic group including combinations of such groups and R⁶ and R⁷ are preferably selected from H or CH₃, and preferably one of R⁶ or R⁷ is H and the other is CH₃. In the case where R^{a} and R^{a′} form an aryl or heteroaryl group, R⁵ is absent.

In one embodiment, a preferred O—Cₖ—O group is an aromatic group having the general structure

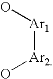

In this preferred structure, Ar₁ and Ar₂ are individually aryl, substituted aryl, or heteroaryl. Examples of useful O—Cₖ—O groups having this general structure include, but are not limited to the following:

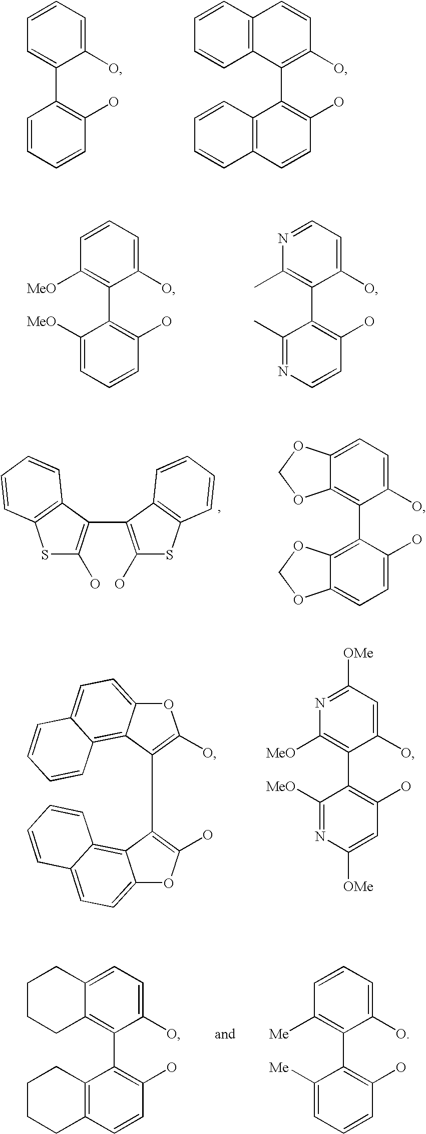

Preferred groups for 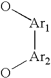
in aspects of the present invention include:

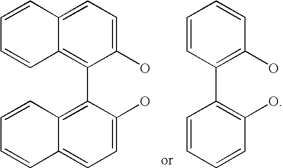

It will be understood by those skilled in the art that these structures may be in any combination of R or S enantiomers, and that both enantiomers may be implemented in the present invention.

In an alternative preferred embodiment, the O—Cₖ—O group is an aliphatic group. Examples of useful aliphatic groups include, but are not limited to, 2,3-butanediol, 1,2-propanediol, 2-phenylethylene glycol, or compounds having the following structures:

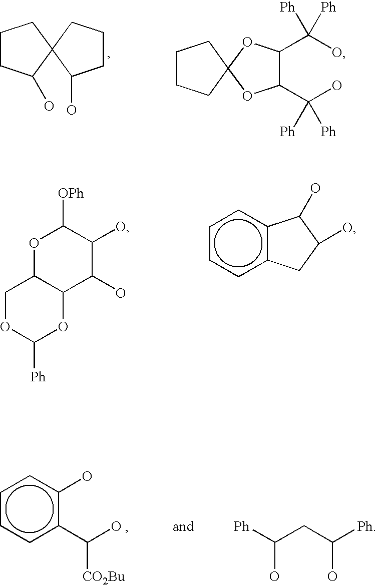

In the general structure above, O—Cₙ—O is preferably a substituted or unsubstituted moiety having the structures:

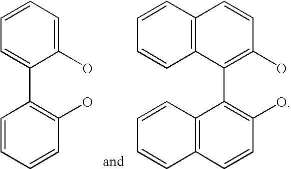

Particularly useful phosphoramidite ligands include various diastereomers of the phosphoramidites having the structures

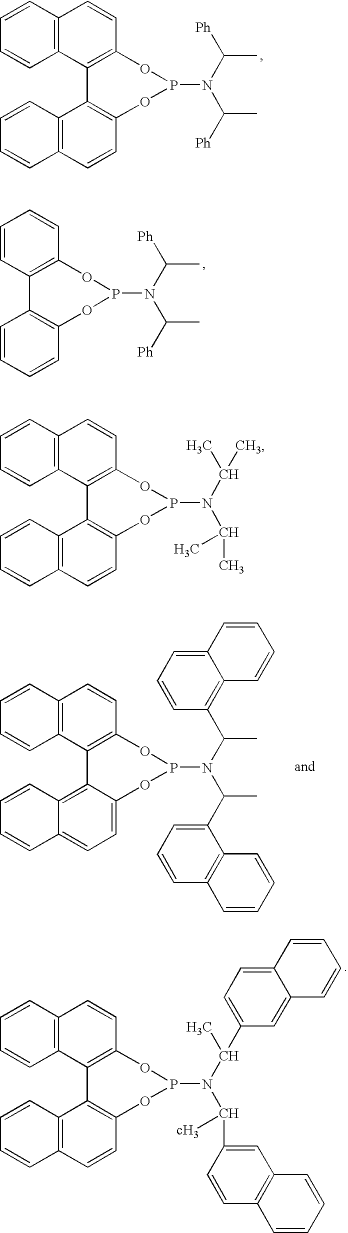

The phosphoramidites of the present invention may be produced using known procedures, such as those described by Alexakis et al. (Alexakis, A. et al., Synlett (2001), 1375), which is incorporated by reference in its entirety herein.

The catalyst precursor and phosphoramidite ligand of the catalyst composition form a catalyst for allylic amination or etherification of achiral or racemic allylic esters in situ (e.g., in the vessel where the allylic amination or etherification is occurring). Alternatively, catalysts according to the present invention may facilitate enatioselective and/or regioselective hydrogenation or transfer hydrogenation reactions, including hydroamination reactions and conjugate addition reactions.

Allylic Amination Reaction

In one embodiment, the present invention is directed to a general method of preparing allylic amines enantioselectively. The method comprises the steps of reacting (a) an achiral or racemic allylic ester, allylic carbonate or allylic halide; and (b) a reactant containing an N—H bond or a salt thereof, excluding lithium salts of N-benzyltosylamides, in the presence of a solvent and a catalyst composition. The catalyst composition may be any catalyst composition that contains a transition metal selected from the group consisting of iridium, rhodium, molybdenum, and tungsten.

The catalyst for the above general reaction preferably comprises (1) a catalyst precursor having the general structure MSXₙ wherein M is the above transition metal; S is a coordinating ligand; X is a counterion; and n is an integer from 0 to 6; and (2) a phosphoramidite ligand having the structure: 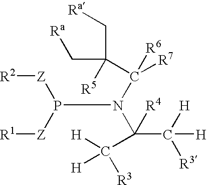
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂),-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)-aromatic group;
R⁵ is absent, H, an optionally substituted C₁-C₁₂ alkyl group or an optionally substituted (CH₂),-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a saturated or unsaturated carbocyclic ring or heterocyclic ring or an aromatic or heteroaromatic ring, R⁵ is absent or is preferably H, R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center.

In a preferred embodiment, the present invention is further directed to methods of preparing allylic amines enantioselectively, wherein the method comprises the step of reacting an achiral or racemic allylic ester, allylic carbonate or allylic halide; and a reactant containing an N—H bond, or a salt thereof, in the presence of a solvent and the above catalyst composition.

As indicated above, one reactant in the method of the present invention is an achiral or racemic allylic ester, an achiral or racemic allylic carbonate, or an achiral or racemic halide. Useful achiral or racemic allylic esters are preferably selected from the group consisting of: Ph-CH═CH—CH₂—OAc, 4-MeO—C₆H₄—CH═CH—CH₂—OAc, 4-NO₂—C₆H₄—CH═CH—CH₂—OAc, 2-MeO—C₆H₄—CH═CH—CH₂—OAc, 2-furyl-CH═CH—CH₂—OAc, n-C₃H₇—CH═CH—CH₂—OAc, Me-CH═CH—CH₂—OAc, n-Pr—CH═CH—CH₂—OAc, i-Pr—CH═CH—CH₂—OAc, and CH₃—CH═CH—CH═CH—CH₂—OAc. As will be appreciated by those skilled in the art, combinations of the above achiral allylic esters may also be implemented in the method of the invention.

Useful achiral or racemic allylic carbonates that may be implemented in the method of the invention include Ph-CH═CH—CH₂—OCOOMe, 4-MeO—C₆H₄—CH═CH—CH₂—OCOOMe, 4-NO₂—C₆H₄—CH═CH—CH₂—OCOOMe, 2-MeO—C₆H₄—CH═CH—CH₂—OCOOMe, 2-furyl-CH═CH—CH₂—OCOOMe, n-C₃H₇—CH═CH—CH₂—OCOOMe, Me-CH═CH—CH₂—OCOOMe, n-Pr—CH═CH—CH₂—OCOOMe, i-Pr—CH═CH—CH₂—OCOOMe, and CH₃—CH═CH—CH═CH—CH₂—OCOOMe. Combinations of the above achiral allylic carbonates may also be implemented.

Notwithstanding the above examples, it will be appreciated by those skilled in the art that other esters and carbonates can be used in the present invention, besides acetate and methyl carbonates shown above. For example, ethyl, t-butyl phenyl, or other suitable aliphatic or aromatic group could replace methyl.

Useful achiral or racemic allylic halides that may be implemented in the method of the invention include Ph-CH═CH—CH₂—X, 4-MeO—C₆H₄—CH═CH—CH₂—X, 4-NO₂—C₆H₄—CH═CH—CH₂—X, 2-MeO—C₆H₄—CH═CH—CH₂—X, 2-furyl-CH═CH—CH₂—X, n-C₃H₇—CH═CH—CH₂—X, Me-CH═CH—CH₂—X, n-Pr—CH═CH—CH₂—X, i-Pr—CH═CH—CH₂—X, and CH₃—CH═CH—CH═CH—CH₂—X, wherein X is selected from a halide atom such as F, Cl, Br, and I.

Generally, useful reagents containing an N—H bond include ammonia, aromatic or aliphatic primary or secondary amines, amides, carbamates, sulfonamides, imides, phosphoramides, imines, silylamines, heterocycles, and combinations and salts thereof. More specific examples of useful reagents with N—H bonds that may be used in the method of the invention include ammonia, aromatic or aliphatic primary or secondary amines such as substituted or unsubstituted anilines, diphenylmethylamine, benzylamines, 4-methoxybenzylamine; primary alkylamines such as n-hexylamine and allyl amines; secondary cyclic amines such as pyrrolidine, piperidine, and morpholine; acyclic secondary amines such as diethylamine; and non-amine substrates like Boc₂NLi, LiN(CHO)₂, benzphenone imine, and tosylamide. Suitable mixtures of the above amine compounds may also be implemented.

Additional additives, such as metal salts (e.g., copper or zinc salts), metal halides (e.g., copper chloride or zinc chloride), 1,4-diazabicyclo(2.2.2)octane (DABCO), and the like, as well as various combinations of these, may also be implemented in the present invention. Particularly useful additives are those that function as bases, including, but not limited to triethylamine or other tertiaryl alkylamines, cyclic tertiaryamines such as 1,4-diazabicyclo(2.2.2)octane (DABCO), and imines such as diazabicycloundecane.

In general, the reaction conditions for the amination method of the present invention include reaction temperatures ranging from 20 to 60° C., and reaction times ranging from 1 to 96 hours. Generally, the ratio of the amounts of phosphoramidite ligand to catalyst precursor is approximately 2:1, and the enantiomeric excess (ee) of said method is typically greater than approximately 70%.

Solvent can influence the reactivity, regioselectivity, and enantioselectivity of the reaction scheme. Useful solvents for the amination reaction include DMF, ethanol, methanol, THF, acetonitrile, CH₃NO₂, DME, CH₂Cl₂, triethylamine, 1,4-dioxane, diethyl ether, toluene, hexane, and combinations thereof. The reactivity at room temperature followed the order DMF, EtOH>MeOH, THF, CH₃CN>CH₃NO₂, DME>CH₂Cl₂, Et₃N>1,4-dioxane, Et₂O, toluene. Reactions in each solvent occur with high regioselectivity (ratio of products 3/4/5=98-94/1-4/0-3) except for those in Et₃N and CH₃NO₂. The enantioselectivity of reactions in different solvents followed the order: THF, Et₂O, DME>toluene, 1,4-dioxane, CH₂Cl₂>Et₃N>DMF, EtOH, CH₃CN>CH₃NO₂>MeOH. Reactions in the polar solvents DMF, EtOH, and MeOH were fast, but lower ee's were observed. Reactions in THF (tetrahydrofuran) displayed the most suitable balance of rate and enantioselectivity.

Etherification Reaction

In one embodiment, the present invention is directed to a general method of preparing allylic ethers enantioselectively. This general method comprising the steps of reacting (a) an achiral or racemic allylic ester, allylic carbonate or allylic halide and (b) a reagent containing a O—H bond or a salt thereof, and (c) a base. The reacting step takes place in a solvent and in the presence of a catalyst composition. The catalyst composition may be any catalyst composition that contains a transition metal selected from the group consisting of iridium, rhodium, ruthenium, molybdenum, and tungsten.

Like the amination reaction, the catalyst for the above general etherification reaction preferably comprises (1) a catalyst precursor having the general structure MSXₙ wherein M is the above transition metal; S is a coordinating ligand; X is a counterion; and n is an integer from 0 to 6; and (2) a phosphoramidite ligand having the structure: 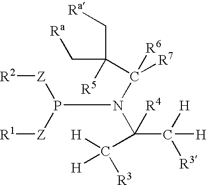
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³ ′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a saturated or unsaturated carbocyclic ring or heterocyclic ring or an aromatic or heteroaromatic ring, R⁵ is absent or is preferably H, R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center.

In a preferred embodiment, the present invention is further directed to methods of preparing allylic ethers enantioselectively, wherein the method comprises reacting an achiral or racemic allylic ester, allylic carbonate or allylic halide and a reagent containing an O—H bond, wherein the reacting step takes place in the presence of the above catalyst composition. The reaction can also be conducted in the presence of an optional additional additive such as metal salts (e.g., copper or zinc salts), metal halides (e.g., copper chloride or zinc chloride), bases such as 1,4-diazabicyclo(2.2.2)octane (DABCO), diazobicycloundecane, as well as various combinations of these to generate the active catalyst or promote desirable reactivity of the reactant having an OH bond, or the salt thereof.

In one embodiment, the achiral allylic ester is preferably an achiral allylic ester or an achiral allylic carbonate. Examples of preferable achiral or racemic allylic acetates include, but are not limited to Ph-CH═CH—CH₂—OAc, 4-MeO—C₆H₄—CH═CH—CH₂—OAc, 4-NO₂—C₆H₄—CH═CH—CH₂—OAc, 2-MeO—C₆H₄—CH═CH—CH₂—OAc, 2-furyl-CH═CH—CH₂—OAc, n-C₃H₇—CH═CH—CH₂—OAc, Me-CH═CH—CH₂—OAc, n-Pr—CH═CH—CH₂—OAc, i-Pr—CH═CH—CH₂—OAc, CH₃—CH═CH—CH═CH—CH₂—OAc, and the like. Combinations of the above achiral allylic acetates may also be employed in the etherification reaction.

Examples of preferable achiral or racemic allylic carbonates that may be implemented in the method of the invention include Ph-CH═CH—CH₂—OCOOR₂, 4-MeO—C₆H₄—CH═CH—CH₂—OCOOR₂, 4-NO₂—C₆H₄—CH═CH—CH₂—OCOOR₂, 2-MeO—C₆H₄—CH═CH—CH₂—OCOOR₂, 2-furyl-CH═CH—CH₂—OCOOR₂, n-C₃H₇—CH═CH—CH₂—OCOOR₂, Me-CH═CH—CH₂—OCOOR₂, n-Pr—CH═CH—CH₂—OCOOR₂, i-Pr—CH═CH—CH₂—OCOOR₂, CH₃—CH═CH—CH═CH—CH₂—OCOOR₂, and the like. In each formula above, R₂ is a methyl or ethyl group. However, notwithstanding the above examples, it will be appreciated by those skilled in the art that other esters and carbonates can be used in the present invention, besides acetate and methyl carbonates shown above. For example, ethyl, t-butyl, phenyl, or another suitable aliphatic or aromatic group could replace methyl. As will further be appreciated, combinations of achiral allylic carbonates may also be implemented.

Useful achiral or racemic allylic halides that may be implemented in the method of the invention include Ph-CH═CH—CH₂—X, 4-MeO—C₆H₄—CH═CH—CH₂—X, 4-NO₂—C₆H₄—CH═CH—CH₂—X, 2-MeO—C₆H₄—CH═CH—CH₂—X, 2-furyl-CH═CH—CH₂—X, n-C₃H₇—CH═CH—CH₂—X, Me-CH═CH—CH₂—X, n-Pr—CH═CH—CH₂—X, i-Pr—CH═CH—CH₂—X, and CH₃—CH═CH—CH═CH—CH₂—X, wherein X is selected from a halide atom such as F, Cl, Br, and I.

Useful reagents that contain an O—H bond include alkoxides, phenoxides, siloxides, carboxylates, phosphates, alcohols, phenols, silanols, carboxylic acids, phosphorus-containing acids, and salts thereof. Specific examples of useful reagents containing an O—H bond that may be employed in the etherification method of the invention include 2-MeC₆H₄OLi, 4-MeC₆H₄OLi, 4-MeOC₆H₄OLi, 3-MeOC₆H₄OLi, 3-PhC₆H₄OLi, 2-PhC₆H₄OLi, 3-Me₂NC₆H₄OLi, 3,4-(OCH₂O)C₆H₃OLi, 2,4-Me₂C₆H₃OLi, 2,4,6-Me₃C₆H₃OLi, 4-BrC₆H₄ONa, 4-ClC₆H₄ONa, 4-Br,3-MeC₆H₄ONa, 4-CF₃C₆H₄ONa, PhOLi, PhONa, as well as salts of these.

The etherification reaction may be carried out in the presence of an optional base. Examples of useful bases in the etherification method of the invention include 1,4-diazabicyclo(2.2.2)octane (DABCO), triethylamine, isopropyldiethylamine, ethyl dimethylamine, metal hydrides, amides, alkoxides, carbonates, and phosphates. Examples of useful solvents include DMF, ethanol, methanol, THF, acetonitrile, CH₃NO₂, DME, CH₂Cl₂, triethylamine, 1,4-dioxane, diethyl ether, toluene, hexane, and combinations thereof, as well as aqueous mixtures thereof.

Like the allylic amination reaction described above, the general reaction conditions for the etherification method of the present invention include reaction temperatures ranging from about 20 to 60° C., and reaction times ranging from about 1 to 96 hours or longer.

Generally, the ratio of the amounts of phosphoramidite ligand to catalyst precursor is approximately 2:1, and the enantiomeric excess (ee) of the method is typically greater than approximately 70%.

Choice of base and solvent and matching of the phenoxide nucleophile with the appropriate allylic carbonate derivative were crucial to observe high yields, regioselectivities, and enantioselectvities for formation of the major product.

Allylic Alkylation Reaction

In another embodiment, the present invention is directed to a general method of preparing products with new carbon-carbon bonds by addition of enolate or other stabilized cabanionic reagents to achiral or racemic allylic esters or halides in the presence of optional additives. This general method comprising the steps of reacting (a) an achiral or racemic allylic ester, allylic carbonate or allylic halide and (b) a compound derived from a 1,3-dicarbonyl compound, cyanoester or carbonyl compound with a P-sulfone or phosphate, or the corresponding neutral reagent that is converted into a carbanionic reagent in the presence of base and (c) an optional additive, such as a metal salt or other halide additive. The reacting step takes place in a solvent and in the presence of a catalyst composition. The catalyst composition may be any catalyst composition that contains a transition metal selected from the group consisting of iridium, rhodium, ruthenium, molybdenum, and tungsten.

Like the amination reaction, the catalyst for the above general allylic alkylation reaction preferably comprises (1) a catalyst precursor having the general structure MSXₙ wherein M is the above transition metal; S is a coordinating ligand; X is a counterion; and n is an integer from 0 to 6; and (2) a phosphoramidite ligand having the structure: 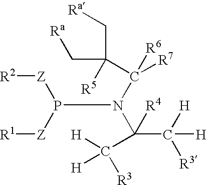
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, amnoalcohol, aminothiol or alcoholthiol group;
R^{3′} and R³ are each independently H, an optionally substituted C₁-C₁₂ alkyl group or an optionally substituted (CH₂)-aromatic group with the proviso that R^{3′} and R³ are not both H, or together R³ ′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a saturated or unsaturated carbocyclic ring or heterocyclic ring or an aromatic or heteroaromatic ring, R⁵ is absent or is preferably H, R⁶ and R¹ are preferably H or CH₃; and
Each n is independently 0, 1, 2, 3, 4, 5 or 6 and wherein at least one of the carbon atoms attached to the nitrogen of the phosphoramidite group is a chiral center.

In a preferred embodiment, the present invention is further directed to methods of preparing allylic alkylation products enantioselectively, wherein the method comprises reacting an achiral or racemic allylic ester, allylic carbonate or allylic halide and a carbanionic reagent derived from a 1,3-dicarbonyl compound, cyanoester, or carbonyl compound with a P-sulfone or phosphate, or the corresponding neutral reagent that is converted into a carbanionic reagent in the presence of base, wherein the reacting step takes place in the presence of the above catalyst composition. The reaction can also be conducted in the presence of an optional additional additive such as metal salts (e.g., copper or zinc salts), metal halides (e.g., copper chloride or zinc chloride), bases such as 1,4-diazabicyclo(2.2.2)octane (DABCO), diazobicycloundecane, as well as various combinations of these to generate the active catalyst or promote desirable reactivity of the reactant having a C—H bond, or the salt thereof.

In one embodiment, the achiral allylic ester is preferably an achiral allylic ester or an achiral allylic carbonate. Examples of preferable achiral or racemic allylic acetates include, but are not limited to Ph-CH═CH—CH₂—OAc, 4-MeO—C₆H₄—CH═CH—CH₂—OAc, 4-NO₂—C₆H₄—CH═CH—CH₂—OAc, 2-MeO—C₆H₄—CH═CH—CH₂—OAc, 2-furyl-CH═CH—CH₂—OAc, n-C₃H₇—CH═CH—CH₂—OAc, Me-CH═CH—CH₂—OAc, n-Pr—CH═CH—CH₂—OAc, i-Pr—CH═CH—CH₂—OAc, CH₃—CH═CH—CH═CH—CH₂—OAc, and the like. Combinations of the above achiral allylic acetates may also be employed in the etherification reaction.

Examples of preferable achiral or racemic allylic carbonates that may be implemented in the method of the invention include Ph-CH═CH—CH₂—OCOOR₂, 4-MeO—C₆H₄—CH═CH—CH₂—OCOOR₂, 4-NO₂—C₆H₄—CH═CH—CH₂—OCOOR₂, 2-MeO—C₆H₄—CH═CH—CH₂—OCOOR₂, 2-furyl-CH═CH—CH₂—OCOOR₂, n-C₃H₇—CH═CH—CH₂—OCOOR₂, Me-CH═CH—CH₂—OCOOR₂, n-Pr—CH═CH—CH₂—OCOOR₂, i-Pr—CH═CH—CH₂—OCOOR₂, CH₃—CH═CH—CH═CH—CH₂—OCOOR₂, and the like. In each formula above, R₂ is a methyl or ethyl group. However, notwithstanding the above examples, it will be appreciated by those skilled in the art that other esters and carbonates can be used in the present invention, besides acetate and methyl carbonates shown above. For example, ethyl, t-butyl, phenyl, or another suitable aliphatic or aromatic group could replace methyl. As will farther be appreciated, combinations of achiral allylic carbonates may also be implemented.

Useful achiral or racemic allylic halides that may be implemented in the method of the invention include Ph-CH═CH—CH₂—X, 4-MeO—C₆H₄—CH═CH—CH₂—X, 4-NO₂—C₆H₄—CH═CH—CH₂—X, 2-MeO—C₆H₄—CH═CH—CH₂—X, 2-furyl-CH═CH—CH₂—X, n-C₃H₇—CH═CH—CH₂—X, Me-CH═CH—CH₂—X, n-Pr—CH═CH—CH₂—X, i-Pr—CH═CH—CH₂—X, and CH₃—CH═CH—CH═CH—CH₂—X, wherein X is selected from a halide atom such as F, Cl, Br, and I.

Useful enolates include zinc enolates of dialkyl malonates, alkyl or aryl dialkylmalonates, cyanoesters, β-ketoesters, malononitrile, β-ketosulfones, azlactones, 1,3-diketones and the like.

The allylic alkylation reaction may be carried out in the presence of an optional base. Examples of useful bases in the etherification method of the invention include 1,4-diazabicyclo(2.2.2)octane (DABCO), triethylamine, isopropyldiethylamine, ethyl dimethylamine, metal hydrides, amides, alkoxides, carbonates, and phosphates. Examples of useful solvents include DMF, ethanol, methanol, THF, acetonitrile, CH₃NO₂, DME, CH₂Cl₂, triethylamine, 1,4-dioxane, diethyl ether, toluene, hexane, and combinations thereof, as well as aqueous mixtures thereof. The allylic alkylation reaction may be carried out in the presence of an optional halide additive, such as ZnF₂, ZnCl₂, or CsF.

Like the allylic amination reaction described above, the general reaction conditions for the allylic alkylation method of the present invention include reaction temperatures ranging from about 20 to 60° C., and reaction times ranging from about 1 to 96 hours or longer. Generally, the ratio of the amounts of phosphoramidite ligand to catalyst precursor is approximately 2:1, and the enantiomeric excess (ee) of the method is typically greater than approximately 70%.

Choice of base and solvent and matching of the carbon nucleophile with the appropriate allylic carbonate derivative were crucial to observe high yields, regioselectivities, and enantioselectvities for formation of the major product.

The present invention also relates to methods for catalyzing the addition of hydrogen to an olefin using a catalyst according to the present invention to form a reduced, non-racemic chiral product.

In this method a hydrogenation reaction occurs by reacting hydrogen with an olefin compound, a ketone containing compound or an imine containing compound, the reacting step taking place in a solvent and in the presence of a catalyst composition, the catalyst composition comprising (1) a catalyst precursor having the general structure M′SₘXₖ wherein M′ is a transition metal selected from the group consisting of iridium, rhodium or ruthenium; S is a coordinating ligand; X is a counterion; m is an integer from 0 to 6 and k is an integer from 0 to 6; and (2) a phosphoramidite ligand L having the structure: 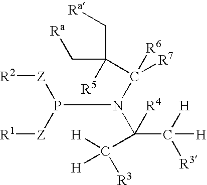
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, amnoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)i-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂),-aromatic group, with the proviso that R⁵, R⁶ and R⁷ cannot simultaneously be H, and when R^{a} and R^{a′}, together with the carbon to which they are attached, form a carbocyclic ring, a heterocyclic ring or an aromatic or heteroaromatic ring,
R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center under conditions which enantioselectively hydrogenate said olefin, said ketone containing compound or said imine containing compound.

In non-limiting specific embodiments of the present invention, in the hydrogenation aspect of the present invention, a compound with a carbon-carbon, carbon-oxygen or carbon-nitrogen (this should be added above, too) double bond as otherwise described herein is reacted with hydrogen in the presence of a phosphoramidite catalyst according to present invention and an optional Lewis or protic acid in solvent to produce, for example, an alpha- or beta-amino acid derivative, alkyl acetate, alkyl amine, or alkylamine derivative, or alpha-hydroxy ketone or ester from an enol acetate, enamine, enamide or beta-dicarbonyl compound. The reaction may take place with all of the various components, i.e., catalyst precursor, phosphoramidite ligand, unsaturated compound, and optional additive being placed together in a single pot, or alternatively, in more than one step with the catalyst being formed from the catalyst precursor and phosphoramidite ligand as described above (the temperature of the reaction will be dependent upon the reactivity of the individual components utilized and may range from below room temperature to elevated temperature of 100-110° C. or more) followed by the addition of the unsaturated compound first or together with hydrogen. The resulting product can be obtained in high yield, as well as enantioselectivity (ee) which is generally greater than 70%. Unsaturated compounds in this context are reactants (compounds) containing at least one carbon-carbon, carbon-oxygen or one carbon-nitrogen double bond and are used in hydrogenation reactions according to the present invention. An olefin is any compound with a carbon-carbon double bond which can participate in hydrogenation reactions according to the present invention, and includes enamides, enamines, enolacetates, vinylarenes and alpha, beta-unsaturated carbonyl compounds; a compound with a carbon-oxygen double bond includes ketones and beta-keto esters, alpha, beta-unsaturated carbonyl compounds, terminal and internal alkenes, vinylarenes, dienes, eneynes, alpha-beta-unsaturated carbonyl compounds, compounds containing carbon-nitrogen double bonds including ketimines and ketimines with additional functional groups.

Conjugate Addition Reaction

In this method a conjugation reaction occurs by reacting a group of the chemical structure M^{c}R^{d}, where M^{c} is Mg(halide), Zn(halide) or Boron(R^{f})₂, R^{d} is alkyl (preferably C₁-C₁₂, more preferably C₁-C₃ alkyl), aryl, vinyl (preferably C₂-C₁₂, more preferably C₂-C₄ vinyl) or alkynyl (preferably C₂-C₁₂, more preferably C₂-C₄ alkynyl) and R^{f} is an alkyl or alkoxy group with a conjugated compound as otherwise described herein in the presence of a solvent and an optional acid, the reacting step taking place in said solvent in the presence of a catalyst composition, the catalyst composition comprising (1) a catalyst precursor having the general structure M′SₘXⱼ wherein M′ is a transition metal selected from the group consisting of rhodium, copper or silver; S is a coordinating ligand; X is a counterion; n is an integer from 0 to 6 and m is an integer from 0 to 6; and (2) a phosphoramidite ligand L having the structure: 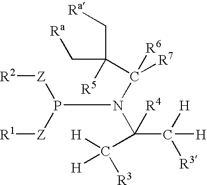
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring or an aromatic or heteroaromatic ring,
R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center, under conditions which enantioselectively conjugate an R^{d} group (add said R^{d} group) to said conjugated compound.

Representative conjugate reactions include the following: 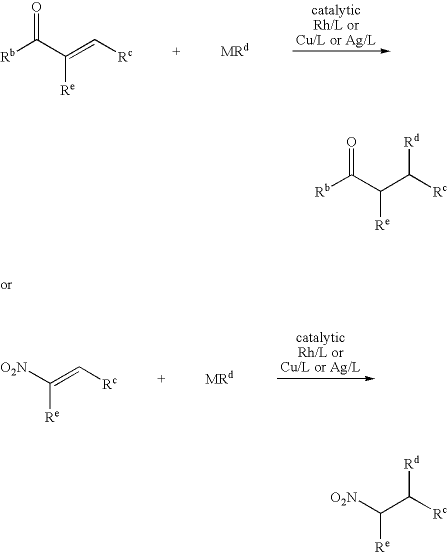
Where R^{b} is alkyl (preferably C₁-C₁₂, more preferably C₁-C₃ alkyl), aryl, alkoxy ((preferably C₁-C₁₂, more preferably C₁-C₃ alkoxy), phenoxy or amino;
R^{c} is alkyl (preferably C₁-C₁₂, more preferably C₁-C₃ allyl), vinyl (preferably C₂-C₁₂, more preferably C₂-C₄ vinyl), aryl or alkynyl (preferably C₂-C₁₂, more preferably C₂-C₄ alkynyl) group;
R^{d} is alkyl (preferably C₁-C₁₂, more preferably C₁-C₃ alkyl), vinyl (preferably C₂-C₁₂, more preferably C₂-C₄ vinyl), aryl, or alkynyl (preferably C₂-C₁₂, more preferably C₂-C₄ alkynyl);
R^{e} is H, alkyl (preferably C₁-C₁₂, more preferably C₁-C₃ alkyl), vinyl (preferably C₂-C₁₂, more preferably C₂-C₄ vinyl), aryl, or alkynyl (preferably C₂-C₁₂, more preferably C₂-C₄ alkynyl); or
any of R^{b}, R^{c}C and R^{e} together are connected to form a cyclic compound;
M^{c} is Mg(halide), Zn(halide) or Boron(R^{f})₂; and
R^{f} is an alkyl or alkoxy group.

### EXAMPLES

The following examples are used to illustrate the present invention.

We conducted the first highly enantioselective aminations and etherifications of allylic carbonates with phosphoramidite ligand L1 containing a binaphtholate unit and a bis-phenethylamino group. We later showed that the active catalyst in these reactions is generated by cyclometalation at one methyl group of the phenethylamino group. This cyclometallation breaks the C₂ symmetry of the ligand and generates a product with only C₁ symmetry. With the information that the active catalyst is generated by cyclometallation induced by a basic reagent, we increased the scope of the process to encompass more weakly basic nucleophiles, such as aromatic amines. To do so, we conducted reactions with catalytic amounts of an aliphatic amine to induce cyclometallation or conducted the catalytic process after activation of the precatalyst with a volatile aliphatic amine.

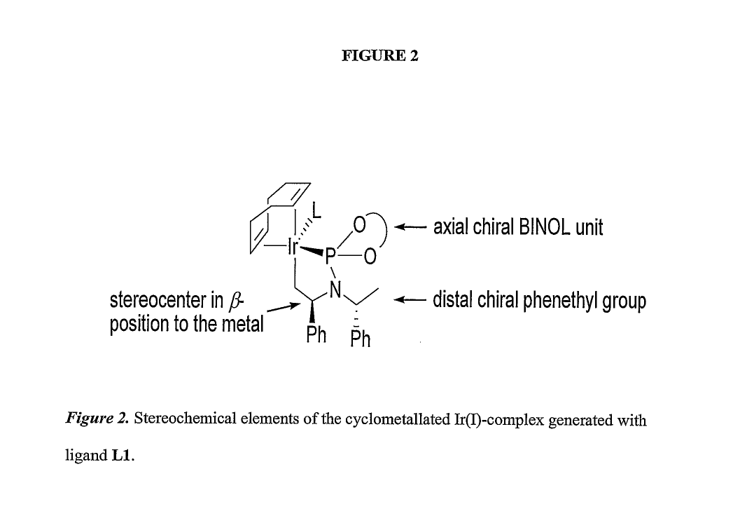

In principle, this information on the cyclometallation of the catalyst should also allow one to prepare new ligands with structures chosen more rationally than the original one. Moreover, the cyclometallated structure provides a platform for studying the origin of the effects of the different stereochemical elements of the ligand and the interconnections between these elements and on enantioselectivity. The cyclometallated catalyst contains one stereocenter remote from the metal, one stereocenter at a carbon β to the metal, and an axial chirality at the binaphthyl unit. Different diastereomers of the ligand can be prepared in a straightforward manner to test the origins of enantioselectivity. The relative importance of the different stereochemical elements and the basis for the difference in activity between catalysts generated from different diastereomeric ligands was not clear from the initial studies of the enantioselective iridium-catalyzed process or even after identifying the structure of the cyclometalated species. See FIG. 2.

We have conducted detailed studies on the effect of the remote substituents, the origins of the difference in reactivity of the different diastereomers, and studies that evaluate the effect of eliminating each of the resolved stereochemical elements. These studies have 1) led to a family of new C₁-symmetric phosphoramidite ligands of the present invention that reveal the importance of different structural features of the ligands and the effect of the interplay between these features on reaction rate and enantioselectivity; 2) shown an unexpected origin of the reactivity of diastereomeric catalysts, and 3) led us to design a ligand that reacts with equally high enantioselectivity as the original ligand, but which contains one phenethylamino substituent as the sole resolved stereochemical element in the entire catalyst structure. Because phenethylamine is an inexpensive optically active building block, these studies uncover a practical catalyst for allylic substitution in concert with concepts that further advance efforts to design catalysts for enantioselective transformations which are cost efficient and relatively easy to synthesize.

Results and Discussion

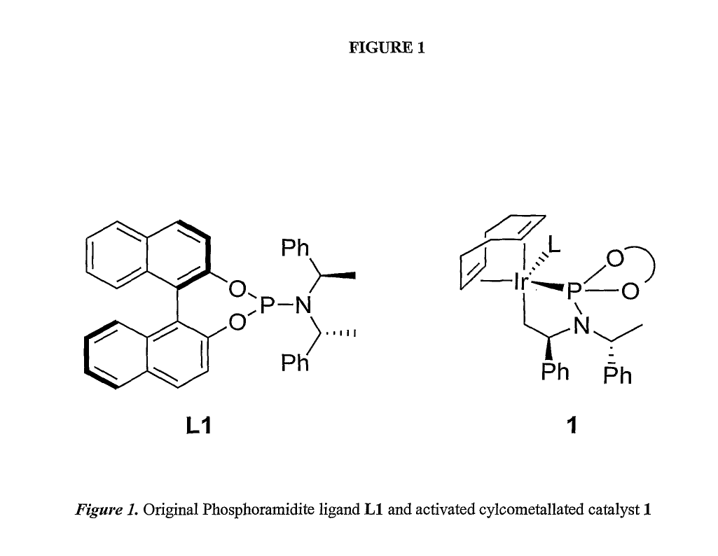

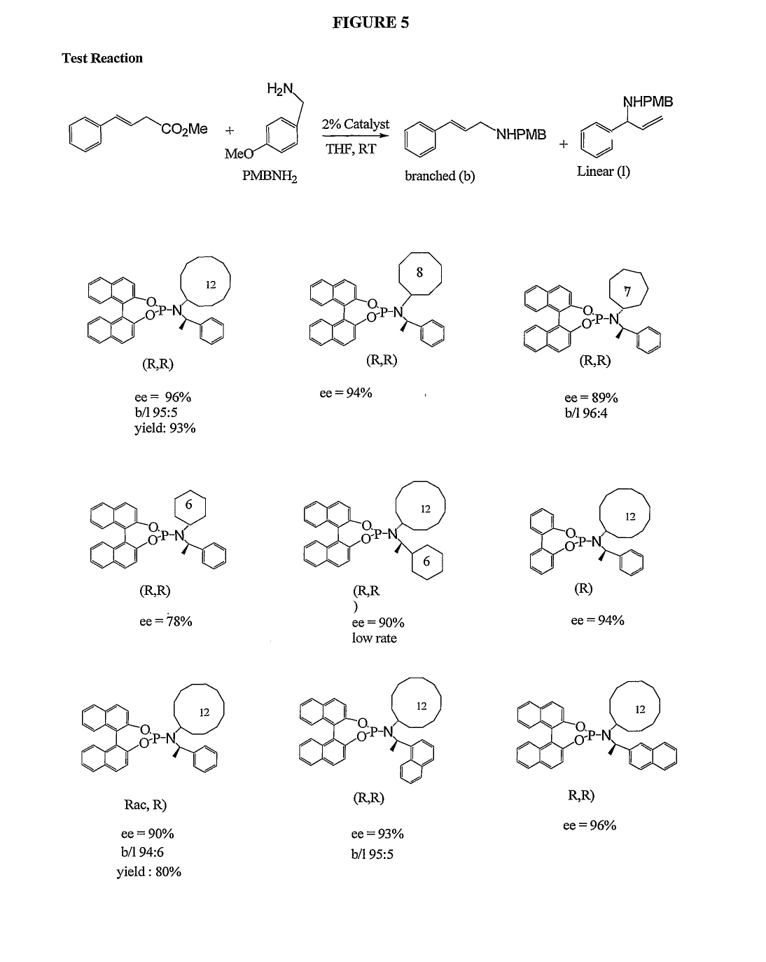

Ligand design. The structure of the product from cyclometallation of ligand L1 contains a distal phenethyl group (see FIG. 1). Thus, we conducted studies to determine if this group could be replaced by an achiral substituent that would impart steric and electronic properties to the coordination sphere that would mimic those of a phenethyl group. To do so, we prepared a series of ligands with cyclic and acyclic aliphatic and benzylic groups at this distal position of the metallocyclic structure. A number of ligands are shown in Table 1 and FIG. 5. The amines for this ligand synthesis were either available commercially or were prepared by simple reductive amination of the appropriate cyclic or acyclic ketones and the chiral phenethylamine. We focused on the synthesis of ligands with distal substituents that lacked methyl groups. The absence of methyl groups would prevent competitive cyclometallation at the methyl group of the phenethyl substituent and at the achiral substituent on nitrogen.

Ligand and Catalyst Preparation. All of the phosphoramidite ligands were prepared following a protocol reported by Alexakis and coworkers, (Alexakis, A. et al., Synlett (2001) for the preparation of C₂-symmetric phosphoramidites. This method simply involves quenching of a lithium amide with PCl₃ and treatment of the aminodichlorophosphine with a diol. All ligands in this study were isolated in pure form and characterized by ¹H and ¹³C NMR spectroscopy. The ligand L6 was prepared on a 2 g scale.

The catalysts were generated by addition of 1 equiv of the ligand to 0.5 equiv. [Ir(COD)Cl]2 to form [Ir(COD)Cl(L)] (1), and heating the combination of metal and ligand with 10 equiv of propylamine at 50° C. in a screw capped vial to generate the cyclometallated species. We have shown with ligand L3 that this procedure generates one equivalent of the trigonal bipyramidal complex analogous to 1 that is ligated by one cyclometallated and one monodentate phosphoramidite ligand and one equivalent of [Ir(COD)Cl]₂. This air and moisture stable yellow solid that is a 1:1 mixture of these two species was isolated after evaporation of the solvent and propylamine.

³¹P NMR spectra of the crude solutions generated upon heating of complexes with L3-L12 indicated that the complexes with ligands that lack methyl groups on the amino substituents undergo cyclometallation to form complexes that are analogous to 1. The formation of analogous cyclometallated complexes was revealed by the appearance of two doublets in the ³¹P NMR spectrum after heating with propylamine. Diastereomers could be generated by cyclometallation at the phenethyl group and selectively at one diastereotopic methyl group of the isopropyl substituent or by reaction at both methyl groups of the isopropyl substituent. Because several products were formed, we were unable to clearly identify the products in the NMR spectra of the crude reactions, and we were unable to isolate a single species in pure form. In either case, the formation of diastereomeric products confirmed our hypothesis that competitive cyclcometallation could occur at the achiral substituent if it contained methyl groups P to nitrogen.

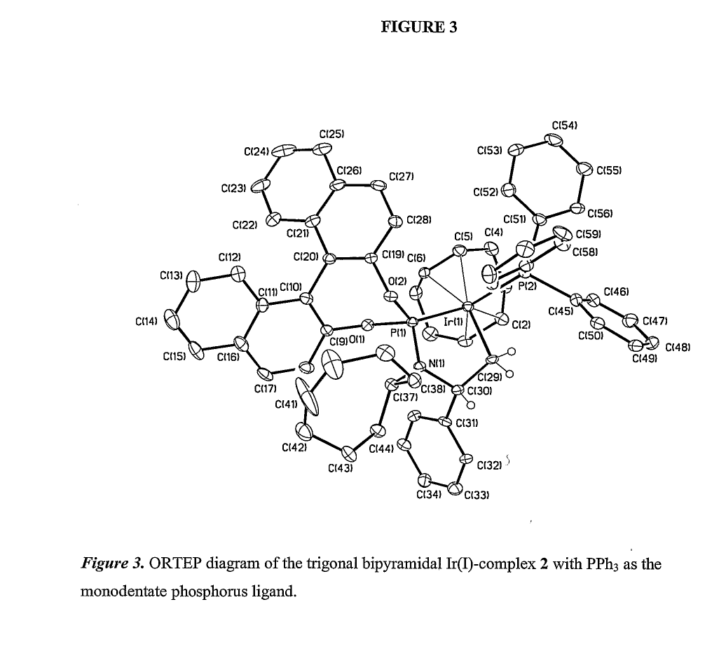

The complex 2 was generated by cyclometallation of the Ir(I) complex of phosphoramidite L5 and replacement of the monodentate phosphoramidite with PPh₃. This complex was characterized by conventional spectroscopic methods and X-ray diffraction. This complex has an analogous composition as the PPh₃ adduct of the original cyclometalated species, except for the achiral group on nitrogen. As depicted in FIG. 3, complex 2 is nearly superimposible with the original structure 1. The cycloalkyl resides in the place of the phenethyl group]. Most important, no cyclcometalation of the ring methylene groups located β to the nitrogen occurred, and a single diasteromer of 2 was formed.

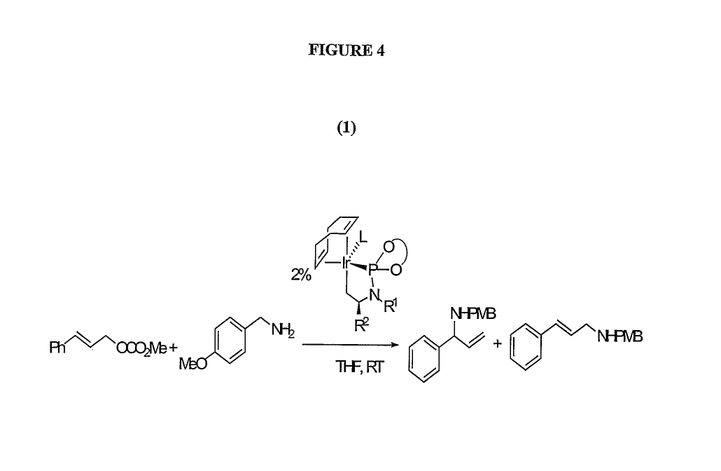

The ability of these ligands to generate catalysts for the formation of the optically active allylic amines from terminal allylic carbonates was probed by conducting the standard reaction of methyl cinnamyl carbonate with p-methoxy benzylamine at room temperature (Eq 1, FIG. 4). These reactions can form the desired branched chiral amine or the linear achiral amine. The enantioselectivity of the branched product, the branched-to-linear ratios (b/1) and the approximate times to >95% conversion of the reactions catalyzed by cyclometalated complexes of the various ligands are shown in Table 1.

From these data, one may extract several trends. First, a comparison of the results in entries 1-7 shows that increased size of the achiral distal substituent attached to the nitrogen may lead to improved enantioselectivities. As the size of the cycloalkyl group was increased from six to seven to eight and then to a 12-membered carbocycle, the enantioselectivity increased. The reaction required a slightly shorter time when catalyzed by the complex derived from the ligand with the largest group. A comparison of these results to those of catalysts derived from ligands with benzylic or fluorenyl substituents showed that the rates were much faster when this substituent was more three-dimensional than planar.

Second, one sees from these data that the reactions with the catalyst containing an isopropyl substituent on nitrogen reacts with reasonably high enantioselectivity and fast rates. Although this catalyst is not the most reactive or more selective, and conclusions from studies of this ligand can be made only tentatively, the results with this ligand do warrant a brief comment. This catalyst is a mixture of species from cyclometalation at the phenethyl group and the isopropyl methyl groups, as described in the previous section. Yet, the compounds generated from cyclometalation at the two substituents are likely to provide the same major enanfromer of the allylic amine. We showed that the reaction of cinnamyl carbonate with benzylamine catalyzed by the complex generated from the phosphoramidite derived from diisopropylamine occurs with a considerable 61% enantioselectivity in favor of the same enantiomer as is formed from the complexes that have undergone cyclometallation at the phenethyl group. Thus, the complexes resulting from cyclometallation of the isopropyl group of the ligand in entry 8, most likely, reacts with the same selectivity and to form the same enantiomer as the catalyst generated from the phosphoramidite with a diisopropylamino group. If so, the composite selectivity of the species generated from cyclometalation at the phenethyl and isopropyl groups would lie between the 61% and 95% ee of the two catalysts. The 89% ee from the composite mixture implies that the species from cyclometalation at the phosphoramidite ligand is somewhat more reactive than that from cyclometalation at the isopropyl group.

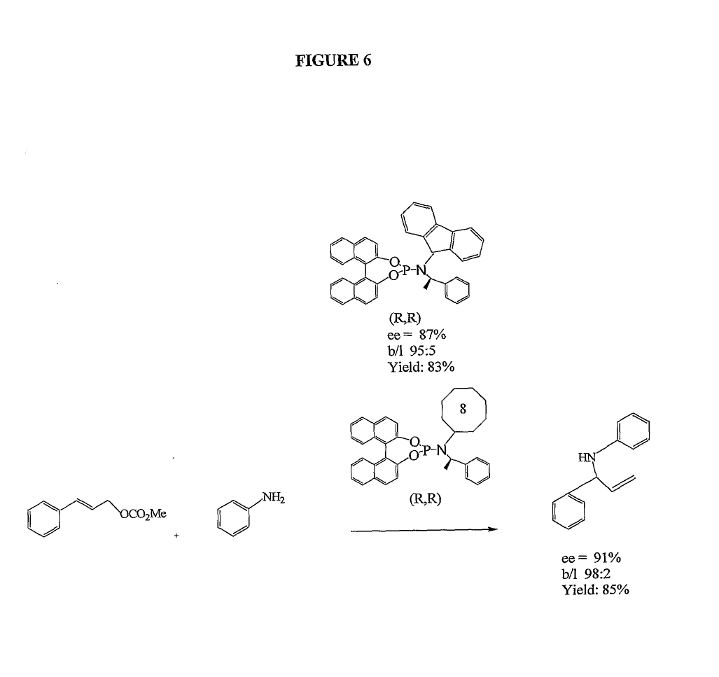

Third, one can conclude that variation of the aryl group on the arylethyl substituent does not dramatically affect the enantioselectivity or regioselectivity but does allow for fine-tuning of enantioselectivity and presumably matching of substrate with catalyst. The highest enantioselectivity was observed with the catalyst derived from the ligand with a 2,6-dichlorophenyl group. The fastest rates were observed with the catalyst derived from the ligand with a 2-methoxy group. The highest branched-to-linear selectivity was measured with the catalyst derived from the ligands with a 2-naphthyl and a 4-fluorophenyl group, although these small differences in an already high selectivity are difficult to clearly identify by ¹H NMR spectroscopy. Also, a number of similar or additional catalysts and their relative enantioselectivities/reactivities are presented in attached FIG. 5. The same reaction with aniline catalyzed by complexes of two different phosphoramidites is shown in FIG. 6.

TABLE 1
Effect of substituents in C₁-symmetric phosphoramidite ligands on the allylic
amination of the above equation.^{a}

| | | | | | reaction |
|---|---|---|---|---|---|
| entry | R¹ | R² | ee^{b} (%) | b/l^{c} | time (h)^{d} |
| 1 | 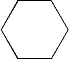 | Ph | 78 | 95:5 | 4 |
| 2 | 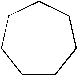 | Ph | 91 | 95:5 | 3 |
| 3 | 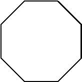 | Ph | 94 | 96:4 | 4 |
| 4 | 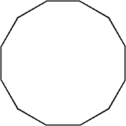 | Ph | 96 | 95:5 | 1.5 |
| 5 | 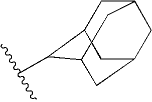 | | 47 | 93:7 | 14 |
| 6 | 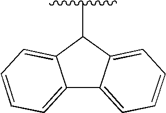 | Ph | 87 | 95:5 | 18 |
| 7 | 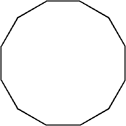 | 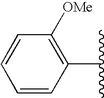 | 97 | 95:5 | 1.5 |
| 8 | | 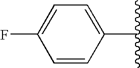 | 97 | 97:3 | 2 |
| 9 | | 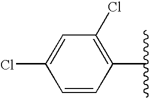 | 98 | 94:6 | 5 |
| 10 | | 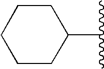 | 90 | | |
| 11 | | 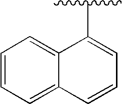 | 93 | 95:5 | 5 |
| 12 | | 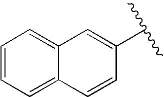 | 96 | 97:3 | 3 |

^{a}Reaction (FIG. 4) were conducted at room temperature on a 1.0 mmol scale in THF (0.5 ml) with a relative mole ratio of carbonate:amine:catalyst of 100:120:2. Enantioselectivities were determined by chiral HPLC. ^{c}Branched to linear ratios were determined by crude ¹H-NMR. ^{d}Reactions were monitored by gas chromatography.

Allylic aminations catalyzed by the complex generated from L6, which contains a cyclododecyl group as the distal substituent at the nitrogen in the metallacycle, occur in yields and enantioselectivities that are as high as those with the original ligand L1, which possesses more elaborate stereochemistry. Results from a series of reactions of aromatic and aliphatic amines with both aliphatic and cinnamyl carbonates catalyzed by the complex generated from L6 are summarized in Table 2. The reactions of benzylamine and two heteroarylmethylamines occurred in good to excellent yields with high branched-to-linear ratios and high enantiomeric excesses with 1 mol % of [Ir(COD)Cl]₂ and 2% of L3. Further, reactions of acyclic secondary amines with this allylic carbonate occurred in excellent yield with very high selectivities, and reactions of aromatic amines occurred in a similar fashion. Finally, the reactions of a linear aliphatic carbonate occurred in equally high yield and selectivity with the simpler ligand L3 as with the original more elaborate L1.

TABLE 2
Amination of allylic carbonates catalyzed by complexes generated from ligand L6.^{a}

| | | | yield^{b} | ee | |
|---|---|---|---|---|---|
| entry | carbonate | amine | (%) | (%) | b/l |
| 1 | 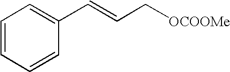 | 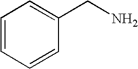 | 93 | 95 | 99:1 |
| 2 | | 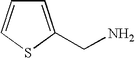 | 78 | 97 | 97:3 |
| 3 | | 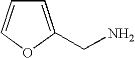 | 86 | 98 | 96:4 |
| 4 | | HNEt₂ | 83 | 98 | 98:2 |
| 5 | | 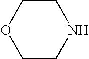 | 93 | 96 | 95:5 |
| 6 | | 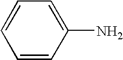 | 92 | 97 | 95:5 |
| 7 | | 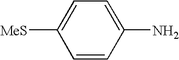 | 94 | 97 | 98:2 |
| 8 | 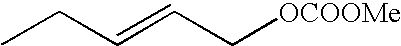 | 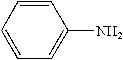 | 80 | 97 | 98:2 |

^{a}Reactions were conducted at room temperature on a 1.0 mmol scale in THF (0.5 ml) with a relative mole ratio carbonate/amine/catalyst of 100:120:2;
^{b}Isolated yields from two independent runs.

A Highly Active and Selective Catalyst from a Single Optically Active Component.

Reactions catalyzed by a complex generated from a ligand related to L6, but containing a biphenolate, instead of binaphtholate, substituent on the phosphorus also occurred in high yield and selectivity. A selective process with this ligand is practical because the ligand is generated from phenethylamine as the only one optically active reagent.

Like the phosphoramidites with binaphtholate groups, phosphoramidite L17 with a biphenolate group was prepared by treatment of N-cyclododecylphenethylamine or the lithium amide with PCl₃, followed by triethylamine and 1,1′-biphenol. The enantiopure ligand was obtained in 60% yield by this two-step synthesis that begins from phenethylamine.

The reaction of benzylamine with methyl cinnamyl carbonate catalyzed by the complex generated from this ligand occurred with rates, yields, regioselectivity and enantioselectivity that are nearly identical to those of the ligand L6 containing a binaphtholate substituent and even the ligand L1 that we used originally and that contains three stereochemical components. As shown in the equation, this reaction occurred in 80% yield with a branched-to-linear ratio of 94:6 and an enantiomeric excess of 94% after only 3 h at room temperature. Other examples are shown in the Table below the equation.

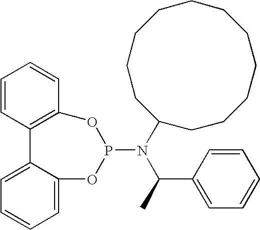

Ligand L17 with a biphenol backbone, an achiral N-alkyl group and one phenethyl group from resolved phenethylamine.

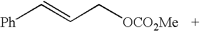

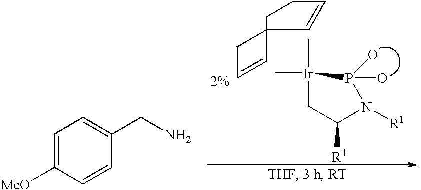

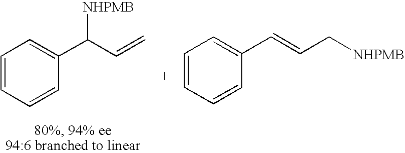

| | | | yield^{b} | ee | |
|---|---|---|---|---|---|
| entry | carbonate | amine | (%) | (%) | b/l |
| 1 | 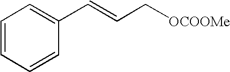 | 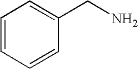 | 86 | 94 | |
| 2 | | 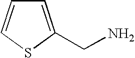 | 83 | 94 | |
| 3 | | 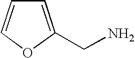 | 84 | 93 | |
| 4 | | HNEt₂ | 72 | nd | |
| 5 | | 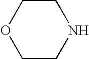 | 85 | nd | |
| 6 | | 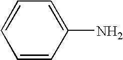 | 96 | 94 | |
| 7 | | 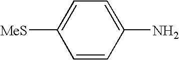 | 81 | 94 | |

Like the reactions with the complexes generated from the other phosphoramidites, we conducted these reactions after inducing cyclometallation. Treatment of [Ir(COD)Cl]₂ with L17 and heating with propylamine led to the active catalyst.

General Procedures for the Enantioselective Allylic Amination.

Catalyst activation with propylamine. [Ir(COD)Cl]₂ (0.020 mmol), phosphoramidite ligand (0.040 mmol) were diluted in 0.2 mL of THF and 0.2 mL propylamine and heated at 50° C. for 20 min. After this time, all volatile materials were evaporated. The yellow solid was diluted in 1.0 mL of THF to generate a stock solution of the activated catalyst.

Catalytic Reaction. In a drybox, amine (1.20 mmol) was added to 0.5 mL of the stock solution of the catalyst (0.020 mmol) in a screw capped vial containing a stirring bar. The vial was sealed with a cap containing a PTFE septum and removed from the drybox. Carbonate (1.00 mmol) was added with a syringe, and the reaction was stirred at room temperature until the carbonate was fully converted, as determined by GC and TLC. The volatile materials were evaporated. The ratio of regioisomers (branched to linear b/1) was determined by ¹H NMR or gas chromatographic analysis of the crude mixture. The crude reaction mixture was purified by flash column chromatography on silica gel (hexanes/ether) to give desired product.

### Allylic Alkylation conducted with O,O′-(R)-(1,1′-Dinaphthyl-2,2′-diyl)-N-cylcododecyl-N′-(R, R)-phenylethylphosphoramidite (L6)

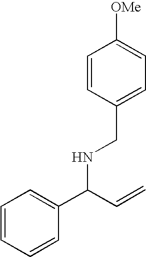

N-(4-methoxybenzyl)-1-phenylprop-2-en-1-amine:$Ohmura, 2002 #8914& The general procedure was followed with cinnamyl methylcarbonate (1.00 mmol) and 4-methoxy benzylamine (0.175 mL, 1.30 mmol). The reactions were conducted at room temperature until the carbonate was fully converted, as determined by GC and TLC. ¹H NMR analysis of the crude reaction mixture indicated the ratio of regioisomers (b/1). The mixture was purified by flash column chromatography on silica gel (hexanes/ether 10:1) to give the title compound. The enantiomeric excess was determined by HPLC [Diacel CHIRALCEL OD-H (0.46 cm×25 cm); hexanes/2-propanol=99.75/0.25; flow rate=0.5 mL/min; detection wavelength=230 nm; TR=26.3 (major), 29.3 (minor) min]. ¹H NMR (400.13 MHz, CDCl₃) δ 7.21-7.38 (m, 7H), 6.86 (d, J=8.4 Hz, 2H), 5.94 (ddd, J=17.2, 10.0, 7.6 Hz, 1H), 5.22 (dt, J=17.2, 1.4 Hz, 1H), 5.11 (ddd, J=10.0, 1.6, 1.2 Hz, 1H), 4.21 (d, J=7.6 Hz, 1H), 3.80 (s, 3H), 3.68 (d, J=13.0 Hz, 1H), 3.64 (d, J=13.0 Hz, 1H), 1.58 (brs, 1H). ¹³C NMR (125.77 MHz, CDCl₃) δ 158.54, 142.84, 141.01, 132.56, 129.29, 128.49, 127.30, 127.14, 115.05, 113.72, 64.99, 55.24, 50.65.

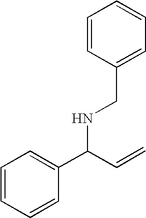

N-(1-Phenyl-2-propenyl)benzylamine: The general procedure was followed with cinnamyl methylcarbonate (0.186 g, 0.97 mmol) and benzylamine (0.130 mL, 1.20 mmol). The reaction was conducted at room temperature for 3.0 h. ¹H NMR analysis of the crude reaction mixture indicated the ratio of regioisomers (b/1) to be 99/1. The mixture was purified by flash column chromatography on silica gel (hexanes/ether 10:1) to give the title compound (0.201 g, 93%). HPLC analysis indicated that the enantiomeric excess of the product was 95% [Diacel CHIRALCEL OD-H (0.46 cm×25 cm); hexanes/2-propanol=99.0/1.0; flow rate=0.5 mL/min; detection wavelength=230 nm; TR=11.96 (major), 13.35 (minor) min]. ¹H NMR (500.13 MHz, CDCl₃) δ 7.30-7.39 (m, 8H), 7.22-7.28 (m, 2H), 5.95 (ddd, J=16.8, 10.0, 7.2 Hz, 1H), 5.23 (dt, J=16.8, 1.6 Hz, 1H), 5.12 (dq, J=10.0, 0.8 Hz, 1H), 4.23 (d, J=7.2 Hz, 1H), 3.75 (d of AB pattern, J=13.4 Hz, 1H), 3.71 (d of AB pattern, J=13.4 Hz, 1H), 1.64 (brs, 1H). ¹³C NMR (125.7 MHz, CDCl₃) δ 142.74, 140.90, 140.38, 128.53, 128.37, 128.15, 127.31, 127.21, 126.89, 115.18, 65.09, 51.26.

1-Phenyl-N-(thiophen-2-ylmethyl)prop-2-en-1-amine. The general procedure was followed with cinnamyl methylcarbonate (0.192 g, 1.00 mmol) and thiophenemethylamine (0.138 mL, 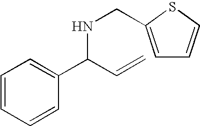
1.34 mmol). The reaction was conducted at room temperature for 3 h. ¹H NMR analysis of the crude reaction mixture indicated the ratio of regioisomers (b/1) to be 97/3. The mixture was purified by flash column chromatography on silica gel (hexanes/ether 10:1) to give the title compound (0.178 g, 78%). HPLC analysis indicated that the enantiomeric excess of the product was 97% [Diacel CHIRALCEL OD-H (0.46 cm×25 cm); hexanes/2-propanol=99.0/1.0; flow rate=0.5 mL/min; detection wavelength=230 nm; TR=11.97 (major), 11.3 (minor) min]. ¹H NMR (500.13 MHz, CDCl₃) δ 7.41-7.33 (m, 5H), 7.32-7.26 (m, 1H), 6.34 (dd, J=3.2, 1.9 Hz, 1H), 6.16 (d, J=3.2, 1H), 5.96 (ddd, J=17.1, 10.1, 7.1 Hz, 1H), 5.25 (dt, J=17.1, 1.24 Hz, 1H), 5.15 (dq, J=10.4, 1.2, 0.7 Hz, 1H), 4.22 (d, J=7.3 Hz, 1H), 3.75 (s, 2H), 1.79 (brs, 1H). ¹³C NMR (125.7 MHz, CDCl₃) δ 153.8, 142.3, 141.7, 140.6, 128.4, 127.3, 127.2, 115.3, 110.0, 106.9, 64.7, 43.6.

N-(Furan-2-ylmethyl)-1-phenylprop-2-en-1-amine. The general procedure was followed with cinnamyl methylcarbonate (0.188 g, 0.98 mmol) and furfurylamine (0.111 mL, 1.20 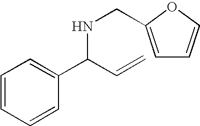
mmol). The reaction was conducted at room temperature for 3.0 h. ¹H NMR analysis of the crude reaction mixture indicated the ratio of regioisomers (b/1) to be 96/4. The mixture was purified by flash column chromatography on silica gel (hexanes/ether 10:1) to give the title compound (0.179 g, 86%). HPLC analysis indicated that the enantiomeric excess of the product was 98% [Diacel CHIRALCEL OD-H (0.46 cm×25 cm); hexanes/2-propanol=99.0/1.0; flow rate=0.5 mL/min; detection wavelength=230 nm; TR=11.35 (major), 11.94 (minor) min]. ¹H NMR (500.13 MHz, CDCl₃) δ 7.41-7.31 (m, 5H), 7.30-7.26 (m, 2H), 6.33 (m, 1H), 6.17 (m, 1H), 5.96 (ddd, J=17.2, 10.4, 7.3 Hz, 1H), 5.25 (dm, J=17.2, 1H), 5.15 (dm, J=10.2, 1H), 4.22 (d, J=7.0 Hz, 2H), 3.75 (s, 2H), 2.20 (brs, 1H). ¹³C NMR (125.7 MHz, CDCl₃) δ 153.7, 142.2, 141.6, 140.5, 128.4, 127.3, 127.2, 115.3, 106.9, 64.6, 43.5. Anal. Calcd. for C₁₄H₁₅NO: C, 78.84; H, 7.09; N, 6.57; O, 7.50. Found: C, 78.60; H, 6.86; N, 6.57.

(1-Phenyl-2-propenyl)diethylamine. The general procedure was followed with cinnamyl methylcarbonate (0.204 g, 1.06 mmol) and diethylamine (0.134 mL, 1.30 mmol). The 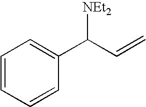
reaction was conducted at room temperature for 3.0 h. ¹H NMR analysis of the crude reaction mixture indicated the ratio of regioisomers (b/i) to be 98/2. The mixture was purified by flash column chromatography on silica gel (hexanes/ether 10:1 to 3:1) to give the title compound (0.166 g, 83%). HPLC analysis indicated that the enantiomeric excess of the product was 98% [Diacel CHIRALCEL OJ (0.46 cm×25 cm); hexanes/diethylamine=99.999/0.001; flow rate=0.4 mL/min; detection wavelength=230 nm; TR=12.11 (major), 11.42 (minor) min]. ¹H NMR (500.13 MHz, CDCl₃) δ 7.19-7.40 (m, 5H), 5.95 (ddd, J=17.2, 10.0, 8.8 Hz, 1H), 5.19 (dd, J=17.2, 1.6 Hz, 1H), 5.12 (dd, J=10.0, 1.6 Hz, 1H), 4.13 (d, J=8.8 Hz, 1H), 2.49-2.63 (m, 4H), 0.98 (t, J=7.2 Hz, 6H). ¹³C NMR (125.7 MHz, CDCl₃) δ 142.96, 139.50, 128.29, 127.82, 126.79, 116.06, 69.46, 42.82, 11.44.

(1-Phenyl-2-propenyl)morpholine. The general procedure was followed with cinnamyl methylcarbonate (0.196 g, 1.02 mmol) and morpholine (0.114 mL, 1.30 mmol). The reaction 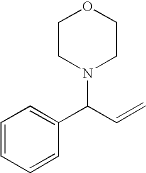
was conducted at room temperature for 3.0 h. ¹H NMR analysis of the crude reaction mixture indicated the ratio of regioisomers (b/1) to be 95/5. The mixture was purified by flash column chromatography on silica gel (hexanes/ether 10:1 to 5:1) to give the title compound (0.192 g, 93%). HPLC analysis indicated that the enantiomeric excess of the product was 96% [Diacel CHIRALCEL OJ (0.46 cm×25 cm); hexanes/2-propanol/diethylamine=98.999/1/0.001; flow rate=0.6 mL/min; detection wavelength=230 nm; TR=12.11 (major), 11.42 (minor) min]. ¹H NMR (500.13 MHz, CDCl₃) δ 7.29-7.36 (m, 4H), 7.22-7.26 (m, 1H), 5.90 (ddd, J=17.2, 10.0, 8.8 Hz, 1H), 5.23 (dd, J=17.2, 1.6 Hz, 1H), 5.10 (dd, J=10.0, 1.6 Hz, 1H), 3.69 (t, J=4.0 Hz, 4H), 3.62 (d, J=8.8 Hz, 1H), 2.43-2.55 (m, 2H), 2.29-2.37 (m, 2H). ¹³C NMR (125.7 MHz, CDCl₃) δ 141.54, 139.70, 128.57, 127.92, 127.24, 116.64, 75.51, 67.12, 51.96.

N-Phenyl-1-phenyl-2-propenylamine. The general procedure was followed with cinnamyl methylcarbonate (0.205 g, 1.06 mmol) and aniline (0.118 mL, 1.24 mmol). The reaction was 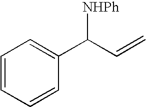
conducted at room temperature for 2.0 h. ¹H NMR analysis of the crude reaction mixture indicated the ratio of regioisomers (b/1) to be 95/5. The mixture was purified by flash column chromatography on silica gel (hexanes/ether 10:1 to 5:1) to give the title compound (0.205 g, 92%). HPLC analysis indicated that the enantiomeric excess of the product was 97% [Diacel CHIRALCEL OJ (0.46 cm×25 cm); hexanes/2-propanol=99.0/1.0; flow rate=0.5 mL/min; detection wavelength=230 nm; TR=22.3 (major), 21.2 (minor) min]. ¹H NMR (500.13 MHz, CDCl₃) δ 7.44-7.24 (m, 5H), 7.20-7.10 (m, 2H), 6.69 (t, J=7.2 Hz, 1H), 6.60 (d, J=8.0 Hz, 2H), 6.04 (ddd, J=16.8, 10.4, 6.4 Hz, 1H), 5.28 (dt, J=17.2, 1.2 Hz, 1H), 5.22 (dt, J=10.0, 1.2 Hz, 1H), 4.94 (t, J=4.4 Hz, 1H), 4.04 (br s, 1H). ¹³C NMR (125.7 MHz, CDCl₃) δ 147.8, 142.4, 139.6, 129.6, 129.1, 127.8, 127.7, 118.2, 115.9, 114.2, 61.1

4-(Methylthio)-N-(1-phenylallyl)benzenamine. The general procedure was followed with cinnamyl methylcarbonate (0.201 g, 1.05 mmol) and aniline (0.159 mL, 1.56 mmol). The reaction was conducted at room temperature for 2.0 h. ¹H NMR analysis of the crude reaction mixture indicated the ratio of regioisomers (b/1) to be 98/2. The mixture was purified by flash column chromatography on silica gel (hexanes/ether 10:1 to 5:1) to give the title compound (0.251 g, 94%). HPLC analysis indicated that the enantiomeric excess of the product was 97% [Diacel CHIRALCEL ODH (0.46 cm×25 cm); hexanes/2-propanol=99.0/1.0; flow rate=0.5 mL/min; detection wavelength=254 nm; TR=23.7 (major), 30.2 (minor) min]. ¹H NMR (500.13 MHz, CDCl₃) δ 7.42-7.27 (m, 6H), 7.19 (d, J=8.9 Hz, 2H), 6.58 (d, J=8.6 Hz, 2H), 6.01 (ddd, J=17.1, 10.5, 5.9 Hz, 1H), 5.32-5.24 (m, 2H), 4.92 (d, J=5.7 Hz, 1H), 2.41 (s, 3H). ¹³C NMR (125.7 MHz, CDCl₃) δ 145.7, 141.3, 138.6, 130.9, 128.5, 127.3, 126.9, 124.3, 115.9, 114.0, 60.5, 18.6. Anal. Calcd. for C₁₆H₁₇NS: C, 75.25; H, 6.71; N, 5.48; S, 12.56. Found: C, 75.56; H, 6.95; N, 5.32.

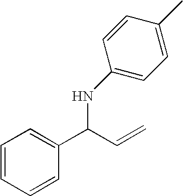

N-(hex-1-en-3-yl)benzenamine. The general procedure was followed with the methylcarbonate derived from (E)-2-hexen-1-ol (0.164 g, 1.04 mmol) and aniline (0.118 mL, 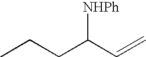
1.30 mmol). The reaction was conducted at room temperature for 2.0 h. ¹H NMR analysis of the crude reaction mixture indicated the ratio of regioisomers (b/1) to be 98/2. The mixture was purified by flash column chromatography on silica gel (hexanes/ether 10:1 to 5:1) to give the title compound (0.146 g, 80%). HPLC analysis indicated that the enantiomeric excess of the product was 97% [Diacel CHIRALCEL ODH (0.46 cm×25 cm); hexanes/2-propanol=99.0/1.0; flow rate=0.5 mL/min; detection wavelength=254 nm; TR=23.7 (major), 30.2 (minor) min]. ¹H NMR (400.13 MHz, CDCl₃) δ 7.22-7.14 (m, 2H), 6.71 (t, J=7.4 Hz, 1H), 6.63 (dd, J=8.0, 0.8 Hz, 2H), 5.77 (ddd, J=17.2, 10.4, 6.0 Hz, 1H), 5.24 (d, J=17.2 Hz, 1H), 5.15 (d, J=10.2 Hz, 1H), 3.85 (q, J=6.4 Hz, 1H), 3.65 (br s, 1H), 1.65-1.55 (m, 2H), 1.53-1.43 (m, 2H), 0.99 (d, J=7.4 Hz, 3H). ¹³C NMR (100.59 MHz, CDCl₃) δ 147.6, 140.1, 129.1, 117.0, 114.9, 113.2, 55.6, 38.0, 19.1, 14.0.

Preparation of C₁-Symmetric Phosphoramidites

General procedure. The procedure is based on the procedure of Alexakis.(Alexakis, A.; Rosset, S.; Allamand, J.; March, S.; Guillen, F.; Benhaim, C. Synlett 2001, 1375). To a solution of PCl₃ (3.0 mmol) in THF (2 ml) at 0° C. were added 5 ml of triethylamine (3.5 mmol) and a solution of the secondary chiral amine (3.0 mmol) in THF (3 ml). This suspension was stirred at RT for 3 h and cooled to 0° C. A solution of BINOL (3.0 mmol) in 6 ml THF was added, and the reaction mixture stirred at room temperature for 18 h. The reaction was diluted with ethyl acetate and filtered through Celite. After removal of the solvent, the resulting white foam was purified by column chromatography (hexanes/ether=10:1).

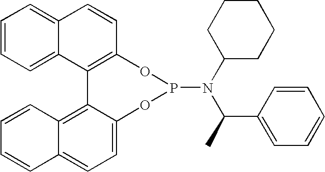

¹H NMR (500.13 MHz, CDCl₃) δ 7.97 (d, J=8.5 Hz, 1H), 7.91 (d, J=8.2 Hz, 1H), 7.85 (d, J=8.2 Hz, 1H), 7.76 (d, J=8.5 Hz, 1H), 7.58 (d, J=7.5 Hz, 1H), 7.53 (d, J=8.8 Hz, 1H), 7.43-7.20 (m, 11H), 4.53 (m, 1H), 2.73 (m, 1H), 1.83 (m, 1H), 1.68 (d, J=6.9 Hz, 3H), 1.60-1.20 (m, 6H), 0.90 (m, 1H), 0.77 (m, 1H), 0.52 (m, 1H). ¹³C NMR (125.7 MHz, CDCl₃) δ 150.43, 150.35, 149.94, 145.5, 132.8 (d, J_{P-C}=1.5 Hz, 1C), 132.6 (d, J_{P-C}=1.5 Hz, 1C), 131.2, 130.3, 130.2, 129.4, 128.2, 128.1, 128.05, 127.04, 127.01, 126.5, 125.9, 125.8, 124.6, 124.3, 124.1, 124.0, 122.4 (d, J_{P-C}=2.5 Hz, 1C, 122.0, 121.6 d, (J_{P-C}=2.3 Hz, 1C), 55.5 (d, J_{P-C}=4.6 Hz, 1C), 53.8, 53.6, 33.8 (d, J_{P-C}=5.5 Hz, 1C), 26.1, 25.3, 24.5, 24.4. ³¹P-NMR (161.9 MHz, CDCl₃) δ 151.7 (s). [α]_{D}²⁵=−178.4 (c 0.790, CHCl₃). Anal. Calcd. for C₃₄H₃₂NO₂P: C, 78.90; H, 6.23; N, 2.71; O, 6.18; P, 5.98. Found: C, 78.72; H, 5.98; N, 2.59.

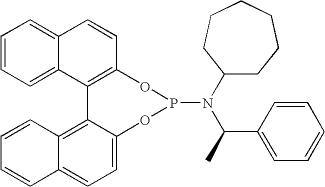

¹H NMR (500.13 MHz, CDCl₃) δ 8.01 (d, J=8.9 Hz, 1H), 7.95 (d, J=8.2 Hz, 1H), 7.88 (d, J=8.2 Hz, 1H), 7.79 (d, J=8.9 Hz, 1H), 7.62 (d, J=7.6 Hz, 1H), 7.58 (d, J=8.5 Hz, 1H), 7.50-7.20 (m, 11H), 4.57 (m, 1H), 3.00 (m, 1H), 1.98 (m, 1H), 1.79 (m, 1H), 1.72 (d, J=7.2 Hz, 3H), 1.70-1.00 (m, 9H), 0.70 (m, 1H). ¹³C NMR (125.7 MHz, CDCl₃) δ 150.3, 150.2, 149.9, 132.8 (d, J_{P-C}=1.5 Hz, 1C), 132.6, 131.2 (d, J_{P-C}=1.5 Hz, 1C), 130.4, 130.2, 129.5, 128.2, 128.1, 128.0, 127.0, 126.98, 126.95, 126.5, 125.9, 125.8, 124.6, 124.3, 124.0, 123.9, 122.35, 122.34, 122.0, 121.64, 121.62, 57.3 (d, J_{P-C}=4.4 Hz, 1C), 54.5, 54.3, 37.0, 35.3, 27.2, 26.8, 25.1, 24.9. ³¹P-NMR (161.9 MHz, CDCl₂) δ 150.7 (s). [α]_{D}²⁵=−142.9 (c 1.055, CHCl₃). Anal. Calcd. for C₃₄H₃₂NO₂P: C, 79.07; H, 6.45; N, 2.63; O, 6.02; P, 5.83. Found: C, 78.82; H, 6.12; N, 2.57.

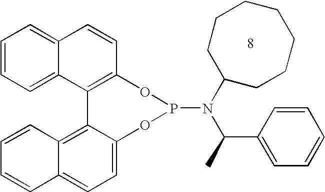

¹H NMR (500.13 MHz, CDCl₃) δ 8.01 (d, J=8.7 Hz, 1H), 7.94 (d, J=8.2 Hz, 1H), 7.86 (d, J=8.2 Hz, 1H), 7.81 (d, J=8.9 Hz, 1H), 7.61 (d, J=7.6 Hz, 1H), 7.58 (d, J=8.5 Hz, 1H), 7.48-7.24 (m, 11H), 4.52 (m, 1H), 3.19 (m, 1H), 1.93-1.77 (m, 2H), 1.72 (d, J=7.0 Hz, 3H), 1.62-0.86 (m, 10H), 0.60 (m, 1H). ¹³C NMR (125.7 MHz, CDCl₃) δ 150.31, 150.3, 150.0. 132.83, 132.82, 132.79, 131.25, 130.5, 130.2, 129.7, 128.4, 128.2, 127.9, 127.0, 126.95, 126.91, 126.88, 126.5, 125.9, 125.8, 124.6, 124.3, 123.9, 123.8, 122.4 (d, J_{P-C}=2.4 Hz, 1C), 122.1, 121.7 (d, J_{P-C}=2.4 Hz, 1C), 55.3 (d, J_{P-C}=3.6 Hz, 1C), 54.8, 54.6, 35.95, 35.98, 26.0, 25.7, 25.6, 24.9, 23.7. ³¹P-NMR (161.9 MHz, CDCl₂) δ 150.9 (s). [α]_{D}²⁵=−169.1 (c 0.725, CHCl₃). Anal. Calcd. for C₃₆H₃₆NO₂P: C, 79.24; H, 6.65; N, 2.57; O, 5.86; P, 5.68. Found: C, 79.14; H, 6.38; N, 2.52.

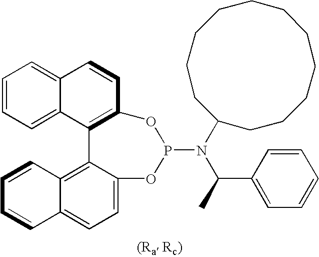

¹H NMR (500.13 MHz, CDCl₃) δ 7.96 (d, J=8.9 Hz, 1H), 7.89 (d, J=8.3 Hz, 1H), 7.82 (d, J=8.3 Hz, 1H), 7.77 (d, J=8.9 Hz, 1H), 7.57 (d, J=7.7 Hz, 1H), 7.51 (d, J=8.5 Hz, 1H), 7.45-7.15 (m, 11H), 4.54 (m, 1H), 3.30 (m, 1H), 1.64 (d, J=7.0 Hz, 3H) 1.38-0.60 (m, 21H). ¹³C NMR (125.7 MHz, CDCl₃) δ 150.2, 150.1, 149.8, 132.9, 132.8 (d, J_{P-C}=1.8 Hz, 1C), 131.3, 130.6, 130.2, 129.9, 128.2, 128.1, 127.9, 127.8, 126.9, 126.7, 125.8, 125.7, 124.6, 124.2, 123.95, 123.91, 122.3, 122.2 (d, J_{P-C}=1.9 Hz, 1C), 121.6, 53.8, 53.7, 50.9 (d, J_{P-C}=5.7 Hz, 1C), 34.6, 32.8, 23.3, 23.2, 22.9, 22.8, 22.7, 22.6 (d, J_{P-C}=3.7 Hz, 1C), 22.1. ³¹P-NMR (161.9 MHz, CDCl₃) δ 151.6 (s). [α]_{D}²⁵=−69.8 (c 0.895, CHCl₃). Anal. Calcd. for C₄₀H₄₄NO₂P: C, 79.84; H, 7.37; N, 2.33; O, 5.32; P, 5.15. Found: C, 79.90; H, 7.15; N, 2.27.

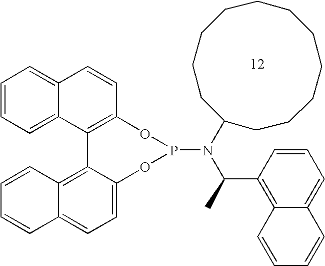

¹H NMR (500.13 MHz, CDCl₃) δ 8.32 (m, 1H), 8.16 (d, J=8.3 Hz, 1H), 8.02 (d, J=8.9 Hz, 1H), 7.95 (d, J=7.8 Hz, 1H), 7.85 (d, J=8.2 Hz, 1H), 7.82 (d, J=8.2 Hz, 1H), 7.74 (d, J=8.6 Hz, 1H), 7.70-7.60 (m, 1H), 7.61-7.50 (m, 3H), 7.45-7.34 (m, 4H), 7.30-7.20 (m, 4H), 5.48 (m, 1H), 3.52 (m, 1H), 1.86 (d, J=7.2 Hz, 3H), 1.70-0.60 (m, 18H). ¹³C NMR (125.7 MHz, CDCl₃) δ 150.3, 150.2, 149.7, 133.8, 132.9, 132.8, 131.3, 130.6, 130.3, 130.2, 129.1, 128.4, 127.9, 127.3, 127.1, 126.9, 126.1, 125.9, 125.8, 125.5, 125.4, 124.6, 124.3, 124.2, 123.9, 123.8, 122.2 (d, J=2.38 Hz, 1C), 121.9, 51.6, 50.8, 50.5, 34.6, 31.95 (d, J=2.6 Hz, 1C), 25.3, 24.9, 24.6, 23.3, 23.0, 22.9, 22.8, 22.7, 21.5. ³¹P-NMR (161.9 MHz, CDCl₃) δ 151.4 (s). [α]_{D}²⁵=−133.3 (c 0.135, CHCl₃). Anal. Calcd. for C₄₄H₄₆NO₂P: C, 81.08; H, 7.11; N, 2.15; O, 4.91; P, 4.75. Found: C, 80.97; H, 6.97; N, 2.08.

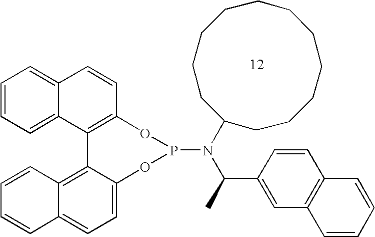

¹H NMR (500.13 MHz, CDCl₃) δ 8.05-7.75 (m, 9H), 7.60-7.50 (m, 3H), 7.45-7.18 (m, 7H), 4.74 (m, 1H), 3.3 7 (m, 1H), 1.76 (d, J=7.1 Hz, 3H), 1.78-0.60 (m, 22H). ¹³C NMR (125.7 MHz, CDCl₃) δ 150.2, 150.18, 149.8, 133.3, 132.9, 132.81, 132.80, 132.5, 131.3, 1230.6, 130.2, 130.0, 128.2, 128.0, 127.9, 127.7, 127.5, 127.1, 127.0, 126.7, 126.0, 125.97, 125.9, 125.8, 125.7, 124.6, 124.3, 123.96, 123.93, 122.3, 122.2, 121.6, 54.1, 54.0, 51.1, 51.0, 32.9, 32.7, 24.3, 23.3, 23.2, 22.8, 22.6, 22.5. ³¹P-NMR (161.9 MHz, CDCl₃) δ 151.3 (s). [α]_{D}²⁵=+26.7 (c 1.050, CHCl₃). Anal. Calcd. for C₄₄H₄₆NO₂P: C, 81.08; H, 7.11; N, 2.15; O, 4.91; P, 4.75. Found: C, 80.80; H, 6.86; N, 2.00.

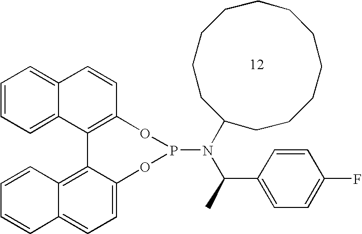

¹H NMR (500.13 MHz, CDCl₃) δ 7.98 (d, J=8.6 Hz, 1H), 7.90 (d, J=8.2 Hz, 1H), 7.85 (d, J=7.6 Hz, 1H), 7.80 (d, J=8.9 Hz, 1H), 7.57-7.49 (m, 3H), 7.43-7.33 (m, 3H), 7.27-7.18 (m, 4H), 7.13-7.06 (m, 2H), 4.54 (m, 1H), 3.30 (m, 1H), 1.74-1.67 (m, 1H), 1.64 (d, J=6.96 Hz, 3H), 1.60-1.51 (m, 2H), 1.37-0.92 (m, 14H), 0.80-0.40 (m, 5H). ¹³C NMR (125.7 MHz, CDCl₃) δ 162.7, 160.8, 150.1, 150.05, 149.7, 132.95 (d, J=2.0 Hz, 1C), 132.8, 131.3, 130.6, 130.3, 129.9, 129.5 (d, J=2.0 Hz, 1C), 129.4, 128.2, 128.0, 127.1, 127.02, 125.9, 125.8, 124.6, 124.3, 123.9, 123.8, 122.14, 122.13 (d, J=2.0 Hz, 1C), 121.6 (d, J=3.1 Hz, 1C), 114.9, 114.8, 53.2, 53.0, 50.9 (d, J=6.7 Hz, 1C), 32.8, 32.7, 24.4, 23.3, 23.2, 22.9, 22.8, 22.6, 22.5, 22.2. ³¹P-NMR (161.9 MHz, CDCl₃) δ 151.5 (s). [α]_{D}²⁵=−92.5 (c 0.845, CHCl₃). Anal. Calcd. for C₄₀H₄₃FNO₂P: C, 77.52; H, 6.99; F, 3.07; N, 2.26; O, 5.16; P, 5.00. Found: C, 77.31; H, 6.72; N, 2.19.

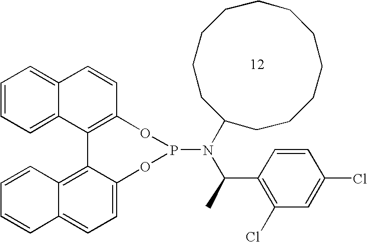

¹H NMR (500.13 MHz, CDCl₃) δ 8.25 (d, J=8.5 Hz, (1H), 8.01 (d, J=8.5 Hz, 1H), 7.94 (d, J=8.2 Hz, 1H), 7.86 (d, J=7.8 Hz, 1H), 7.82 (d, J=8.8 Hz, 1H), 7.57 (d, J=8.8 Hz, 1H), 7.48 (dd, J=8.2, 1.2 Hz, 1H), 7.45 (m, 4H), 7.25-7.22 (m, 4H), 4.98 (m, 1H), 3.50 (m, 1H), 1.72 (m, 1H), 1.48 (m, 2H), 1.40-1.20 (m, 4H), 1.11-0.74 (m, 11H), 0.62 (m, 1H), 0.53 (m, 1H). ¹³C NMR (125.7 MHz, CDCl₃) δ 149.7, 149.6, 149.4, 143.8, 132.9 (d, J=1.5 Hz, 1C), 132.7 (d, J=1.5 Hz, 1C), 132.0, 131.3, 130.7, 130.5, 130.2, 129.5, 129.4, 129.1, 128.2, 127.9, 127.5, 127.0, 126.8, 125.9, 124.7, 124.5, 123.8, 123.7, 121.9 (d, J=2.3 Hz, 1C), 121.8 (d, J=2.3 Hz, 1C), 121.5, 51.5 (d, J=4.6 Hz, 1C), 51.3, 51.1, 31.8, 27.1, 25.1, 24.7, 23.0, 22.9, 22.8, 22.7, 22.4, 21.5. ³¹P-NMR (161.9 MHz, CDCl₃) δ 149.2 (s). [α]_{D.}²⁵=−73.4 (c 1.030, CHCl₃). Anal. Calcd. for C₄₀H₄₂Cl₂NO₂P: C, 71.64; H, 6.31; Cl, 10.57; N, 2.09; O, 4.77; P, 4.62. Found: C, 71.82; H, 6.03; N, 1.95.

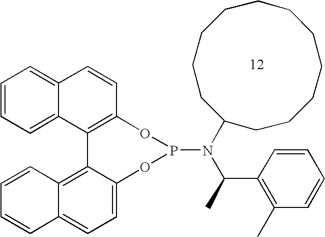

¹H NMR (500.13 MHz, CDCl₃) δ 8.10 (d, J=7.6 Hz, 11H), 8.00 (d, J=8.5 Hz, 1H), 7.94 (d, J=8.5 Hz, 1H), 7.85 (d, J=7.8 Hz, 1H), 7.78 (d, J=8.5 Hz, 1H), 7.45-7.15 (m, 10H), 6.91 (d, J=8.2 Hz, 1H), 5.07 (m, 1H), 3.88 (m, 1H), 3.49 (m, 1H), 1.71 (d, J=7.3 Hz, 3H), 1.50 (m, 2H), 1.40-0.80 (m, 16H). ¹³C NMR (125.7 MHz, CDCl₃) δ 155.3, 150.2, 150.1, 149.8, 135.6, 132.9, 132.8 (d, J=1.6 Hz, 1C), 131.3, 130.6, 130.2, 130.1, 128.2, 128.0, 127.5, 127.0, 126.9, 125.8, 125.7, 124.5, 124.2, 123.9, 123.8, 122.2 (d, J=1.5 Hz, 1C), 122.0, 121.8 (d, J=2.3 Hz, 1C), 120.5, 110.1, 55.2, 51.2 (d, J=3.9 Hz, 1C), 47.8, 47.6, 31.5, 25.1, 24.8, 23.0, 22.97 (d, J=2.2 Hz, 1C), 22.8, 22.7, 22.5, 21.6. ³¹P-NMR (161.9 MHz, CDCl₃) δ 151.2 (s). [α]_{D.9, 22}²⁵=−108.1 (c 1.000, CHCl₃). Anal. Calcd. for C₄₁H₄₆NO₃P: C, 77.94; H, 7.34; N, 2.22; O, 7.60; P, 4.90. Found: C, 77.99; H, 7.60; N, 2.18.

¹H NMR (500.13 MHz, CDCl₃) δ 7.53 (m, 4H), 7.44-7.20 (m, 9H), 4.82 (m, 1H), 3.19 (m, 1H), 1.89 (m, 2H), 1.71-1.63 (m, 1H), 1.59 (d, J=6.87, 3H), 1.491.00 (m, 17H), 0.71 (m, 2H). ¹³C NMR (125.7 MHz, CDCl₃) δ 151.95, 151.89, 151.32, 151.30, 131.26, 131.22, 130.16, 130.13, 129.9, 129.8, 129.1, 128.9 (d, J=1.9 Hz, 1C), 128.4, 127.9, 126.8, 124.45, 124.44, 123.88, 122.44 (d, J=1.9 Hz, 1C), 122.0, 51.8, 50.6, 50.5, 24.1, 23.8, 23.6, 23.5, 23.4, 22.8 (d, J=1.9 Hz, 1C), 22.7, 22.5. ³¹P-NMR (161.9 MHz, CDCl₃) δ 148.1 (s). [α]_{D.9, 22}²⁵=+111.0 (c 0.810, CHCl₃). Anal. Calcd. for C₃₂H₄₀NO₂P: C, 76.62; H, 8.04; N, 2.79; O, 6.38; P, 6.17. Found: C, 76.53; H, 7.78; N, 2.81.

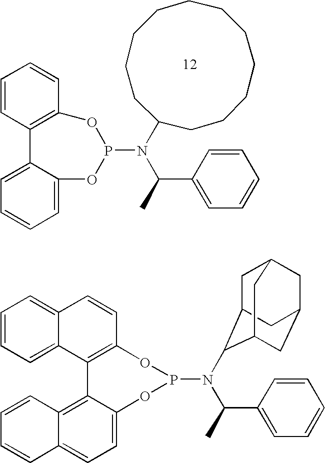

¹H NMR (500.13 MHz, CDCl₃) δ 8.03 (d, J=8.7 Hz, 1H), 7.95 (d, J=8.0 Hz, 1H), 7.82 (d, J=7.6 Hz, 1H), 7.67 (d, J=8.8 Hz, 1H), 7.60-7.53 (m, 3H), 7.47-7.21 (m, 10H), 4.82 (m, 1H), 3.16 (d, J=20.5 Hz, 1H), 2.66 (m, 2H), 2.13 (brs, 1H), 1.99 (brs, 1H), 1.85-1.53 (m, 9H), 1.50 (d, J=6.8 Hz, 1H), 1.40 (m, 1H). ¹³C NMR (125.7 MHz, CDCl₃) δ 151.3, 151.2, 149.8, 142.4, 132.9 (d, J=1.6 Hz, 1C), 132.7, 131.2 (d, J=1.6 Hz, 1C), 130.4 (d, J=1.6 Hz, 1C), 130.1, 129.6, 128.2, 127.97, 127.90, 127.6, 127.2, 127.1, 126.6, 125.9, 125.7, 124.7, 124.3, 124.2, 124.1, 122.5, 122.4, 122.2, 120.5, 120.4, 61.9, 61.8, 53.8, 39.7, 38.9 (d, J=1.5 Hz, 1C), 38.5, 38.4, 35.3, 35.28, 34.26, 34.22, 32.4, 32.1, 31.6, 31.4, 27.5, 27.4, 20.4. ³¹P-NMR (161.9 MHz, CDCl₃) δ 146.0 (s). [α]_{D.9, 22}²⁵=+26.7 (c 1.050, CHCl₃). Anal. Calcd. for C₃₈H₃₆NO₂P: C, 80.12; H, 6.37; N, 2.46; O, 5.62; P, 5.44. Found: C, 79.71; H, 6.60; N, 2.46.

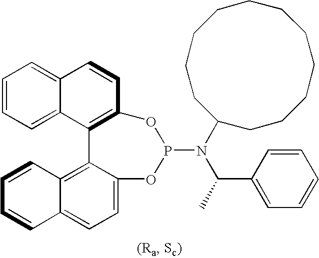

¹H NMR (500.13 MHz, CDCl₃) δ 8.01 (d, J=8.6 Hz, 1H), 7.94 (d, J=8.9 Hz, 1H), 7.90 (d, J=8.1 Hz, 1H), 7.61 (d, J=8.9 Hz, 1H), 7.55 (d, J=8.9 Hz, 1H), 7.50-7.35 (m, 5H), 7.33-7.16 (m, 7H), 4.61 (m, 1H), 3.13 (m, 1H), 1.89 (m, 2H), 1.80-1.54 (m, 3H), 1.50 (d, J=6.8 Hz, 3H), 1.40-0.94 (m, 2H), 0.73 (m, 1H). ¹³C NMR (125.7 MHz, CDCl₃) δ 150.0, 149.9, 149.7, 142.3, 132.8, 131.3, 130.4, 130.2, 129.4, 128.3, 128.2, 128.0, 127.7, 127.0, 126.7, 125.9, 125.8, 124.6, 124.3, 124.0, 123.9, 122.5, 122.3 (d, J=1.53 Hz, 1C), 121.7 (d, J=2.23 Hz, 1C), 51.4, 50.4, 50.2, 34.4 (d, J=10.7 Hz, 1C), 31.6, 25.2, 24.3, 24.0, 23.5, 23.2, 22.8, 22.6, 20.7. ³¹P-NMR (161.9 MHz, CDCl₃) δ 146.9 (s). [α]_{D.}²⁵=−248.7 (c 1.245, CHCl₃). Anal. Calcd. for C₄₁H₄₆NO₃P: C, 79.97; H, 6.88; N, 2.39; O, 5.46; P, 5.29. Found: C, 79.71; H, 6.60; N, 2.46.

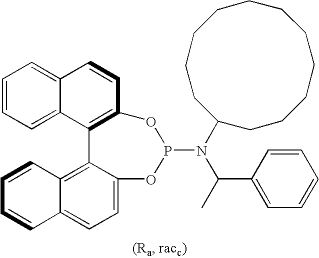

¹H NMR (500.13 MHz, CDCl₃) δ 8.04-7.76 (m, 6H), 7.63-7.50 (m, 4H), 7.50-7.34 (m, 8H), 7.32-7.15 (m, 16H), 4.59 (m, 2H), 3.34 (m, 1H), 3.15 (m, 1H), 1.88 (m, 2H), 1.77-0.85 (m, 41H), 0.78-0.60 (m, 5H), 0.47 (m, 1H), 0.10 (m, 1H). ¹³C NMR (125.7 MHz, CDCl₃) δ 150.2, 150.1, 150.0, 149.97, 149.8, 149.7, 132.9, 132.8, 131.3, 131.2, 130.5, 130.4, 130.2, 129.9, 129.4, 128.3, 128.2, 128.18, 128.1, 128.0, 127.9, 127.8, 127.0, 126.9, 126.8, 126.7, 125.9, 125.85, 125.85, 125.73, 124.6, 124.5, 124.3, 124.2, 123.96, 123.91, 123.86, 122.5, 122.35, 122.25, 122.1, 121.8, 121.7, 121.5, 53.8, 53.7, 51.4, 50.8, 50.5, 50.2, 32.8, 32.7, 31.6, 25.2, 24.4, 24.3, 24.0, 23.5, 23.3, 23.25, 23.2, 23.1, 22.86, 25 22.78, 22.72, 22.61, 22.55, 22.52, 22.0. ³¹P-NMR (161.9 MHz, CDCl₃) δ 151.0 (s), 146.9 (s). [α]_{D.9, 22}²⁵=−152.0 (c 0.665, CHCl₃). Anal. Calcd. for C₄₁H₄₆NO₃P: C, 79.97; H, 6.88; N, 2.39; O, 5.46; P, 5.29. Found: C, 79.71; H, 6.60; N, 2.46.

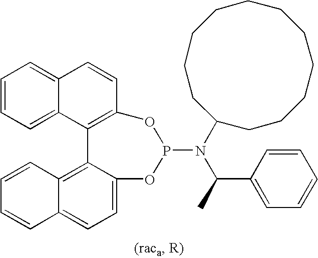

¹H NMR (500.13 MHz, CDCl₃) δ 8.04-7.79 (m, 8H), 7.64-7.50 (m, 5H), 7.49-7.34 (m, 10H), 7.33-7.15 (m, 11H), 4.59 (m, 2H), 3.34 (m, 1H), 3.13 (m, 1H), 1.88 (m, 2H), 1.77-1.43 (m, 14H) 1.37-0.89 (m, 32H), 0.79-0.60 (m, 4H), 0.45 (m, 1H). ¹³C NMR (125.7 MHz, CDCl₃) δ 150.1, 150.08, 149.8, 149.79, 132.9, 132.8, 131.3, 131.3, 130.5, 130.2, 129.9, 129.4, 128.3, 128.2, 128.18, 128.1, 128.0, 127.99, 127.88, 127.80, 127.1, 127.0, 126.8, 126.7, 125.9, 125.89, 125.86, 125.86, 125.76, 124.65, 124.58, 124.34, 124.25, 124.08, 123.92, 122.5, 122.4 (d, J=1.9 Hz, 1C), 122.3, 122.2 (d, J 1=0.9 Hz, 1C), 121.8 (d, J=1.9 Hz, 1C), 53.8, 53.7, 51.4, 51.0, 50.6, 50.4, 34.7, 32.8, 32.7, 31.6, 25.3, 24.7, 24.5, 24.3, 24.0, 23.6, 23.4, 23.3, 23.2, 23.1, 22.9, 22.8, 22.7, 22.6, 22.5, 22.4. ³¹P-NMR (161.9 MHz, CDCl₃) δ 151.0 (s), 146.9 (s). [α]_{D.9, 22}²⁵=+116.3 (c 0.760, CHCl₃). Anal. Calcd. for C₄₁H₄₆NO₃P: C, 79.97; H, 6.88; N, 2.39; O, 5.46; P, 5.29. Found: C, 79.71; H, 6.60; N, 2.46.

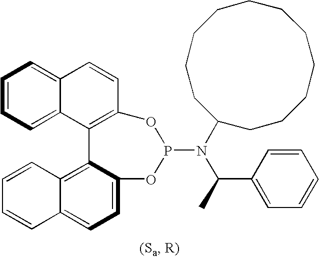

¹H NMR (500.13 MHz, CDCl₃) δ 8.01 (d, J=8.2 Hz, 1H), 7.94 (d, J=8.7 Hz, 1H), 7.90 (d, J=8.2 Hz, 1H), 7.61 (d, J=8.6 Hz, 1H), 7.55 (d, J=8.6 Hz, 1H), 7.60-7.30 (m, 5H), 7.32-7.15 (m, 7H), 4.61 (m, 1H), 3.13 (m, 1H), 1.89 (m, 2H), 1.77-1.54 (m, 3H), 1.50 (d, J=7.1 Hz, 3H), 1.41-0.89 (m, 15H), 0.71 (m, 2H). ¹³C NMR (125.7 MHz, CDCl₃) δ 150.0, 149.9, 149.7, 132.3, 132.8, 131.3, 130.4, 130.2, 129.4, 128.3, 128.2, 128.0, 127.8, 127.1, 126.7, 125.9, 125.8, 124.6, 124.3, 124.0, 123.9, 122.5, 122.3, 121.7, 51.4, 50.5, 50.3, 34.6, 34.4, 31.6, 24.3, 23.9, 23.5, 23.4, 23.3, 22.8, 22.6, 22.5. ³¹P-NMR (161.9 MHz, CDCl₃) δ 146.9 (s). [α]_{D}²⁵=+330.2 (c 1.525, CHCl₃). Anal. Calcd. for C₄₁H₄₆NO₃P: C, 79.97; H, 6.88; N, 2.39; O, 5.46; P, 5.29. Found: C, 79.71; H, 6.60; N, 2.46.

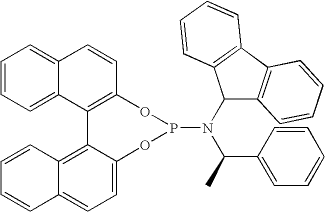

¹H NMR (500.13 MHz, CDCl₃) δ 8.10 (d, J=8.4 Hz, 1H), 7.97 (m, 2H), 7.77 (m, 3H), 7.63 (m, 3H), 7.46 (t, J=7.3 Hz, 1H), 7.40-7.10 (m, 14H), 5.32 (m, 1H) 3.40 (m, 1H), 1.57 (m, 3H). ¹³C NMR (125.7 MHz, CDCl₃) δ 150.0, 149.9, 149.7, 132.3, 132.8, 131.3, 130.4, 130.2, 129.4, 128.3, 128.2, 128.0, 127.8, 127.1, 126.7, 125.9, 125.8, 124.6, 124.3, 124.0, 123.9, 122.5, 122.3, 121.7, 51.4, 50.5, 50.3, 34.6, 34.4, 31.6, 24.3, 23.9, 23.5, 23.4, 23.3, 22.8, 22.6, 22.5. ³¹P-NMR (161.9 MHz, CDCl₃) δ 145.7 (s). [α]_{D}²⁵=+56.2 (c 0.240, CHCl₃). Anal. Calcd. for C₄₁H₃₀ NO₂P: C, 82.12; H, 5.04; N, 2.34; O, 5.34; P, 5.17. Found: C, 81.86; H, 5.05; N, 2.17.

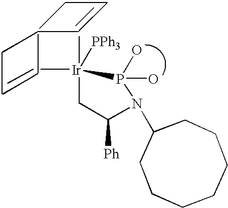

¹H NMR (500.13 MHz, CDCl₃) δ 8.10 (d, J=8.4 Hz, 1H), 7.97 (m, 2H), 7.77 (m, 3H), 7.63 (m, 3H), 7.46 (t, J=7.3 Hz, 1H), 7.40-7.10 (m, 14H), 5.32 (m, 1H) 3.40 (m, 1H), 1.57 (m, 3H). ¹³C NMR (125.7 MHz, CDCl₃) δ 150.0, 149.9, 149.7, 132.3, 132.8, 131.3, 130.4, 130.2, 129.4, 128.3, 128.2, 128.0, 127.8, 127.1, 126.7, 125.9, 125.8, 124.6, 124.3, 124.0, 123.9, 122.5, 122.3, 121.7, 51.4, 50.5, 50.3, 34.6, 34.4, 31.6, 24.3, 23.9, 23.5, 23.4, 23.3, 22.8, 22.6, 22.5. ³¹P-NMR (161.9 MHz, CDCl₃) δ 145.7 (s). [α]_{D}²⁵=+56.2 (c 0.240, CHCl₃). Anal. Calcd. for C₄₁H₃₀NO₂P: C, 82.12; H, 5.04; N, 2.34; O, 5.34; P, 5.17. Found: C, 81.86; H, 5.05; N, 2.17.

1. A compound according to structure: 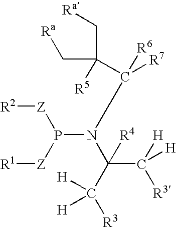
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent, H, an optionally substituted C₁-C₁₂ alkyl group or an optionally substituted (CH₂)ₙ-aromatic or (CH₂)ₙ-heteroaromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H; and
Each n is independently 0, 1, 2, 3, 4, 5 or 6 and wherein at least one of the carbon atoms attached to the nitrogen of the phosphoramidite group is a chiral center.

2. The compound according to claim 1 having the chemical structure: 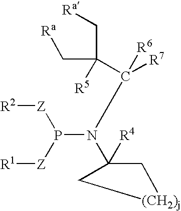
Wherein Z, R¹, R², R⁴, R⁵, R⁶, R⁷, R^{a} and R^{a′} are the same as in claim 1 and j is an integer from 2 to 12.

3. The compound according to claim 1 having the chemical structure: 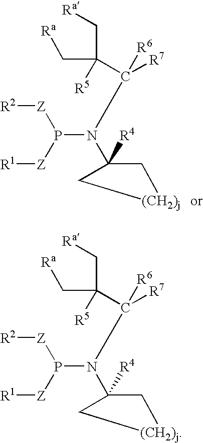
Wherein Z, R¹, R², R⁴, R⁵, R⁶, R⁷, R^{a} and R^{a′} and j are the same as in claims 1 and 2.

4. The compound according to claim 1 wherein R^{a} and R^{a′}, together with the carbon to which they are attached, form a carbocyclic ring, heterocyclic ring or an aromatic or heteroaromatic ring.

5. The compound according to claim 1 wherein R⁵ is absent or is H.

6. The compound according to claim 1 wherein R⁶ and R⁷ are each independently H or CH₃.

7. The compound according to claim 1 wherein the group 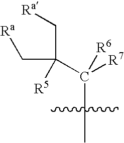
provides a substituted benzyl or naphthylmethyl group connected to the nitrogen atom, such that R⁵ is absent and R⁶ is H and R⁷ is H or CH₃.

8. The compound according to claim 1 wherein R^{3′} and R³ together with the carbon to which they are attached form a carbocyclic group and R⁴ is H or a methyl group.

9. The compound according to claim 1 wherein R¹ and R² are linked and form a biphenyl or binaphthyl group.

10. The compound according to claim 1 wherein the group 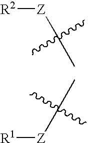
forms an O—Cₖ—O group.

11. The compound according to claim 10 wherein said O—Cₖ—O group is an aliphatic or aromatic diolate.

12. The compound according to claim 10 wherein said 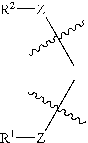
group is selected from the group consisting of:

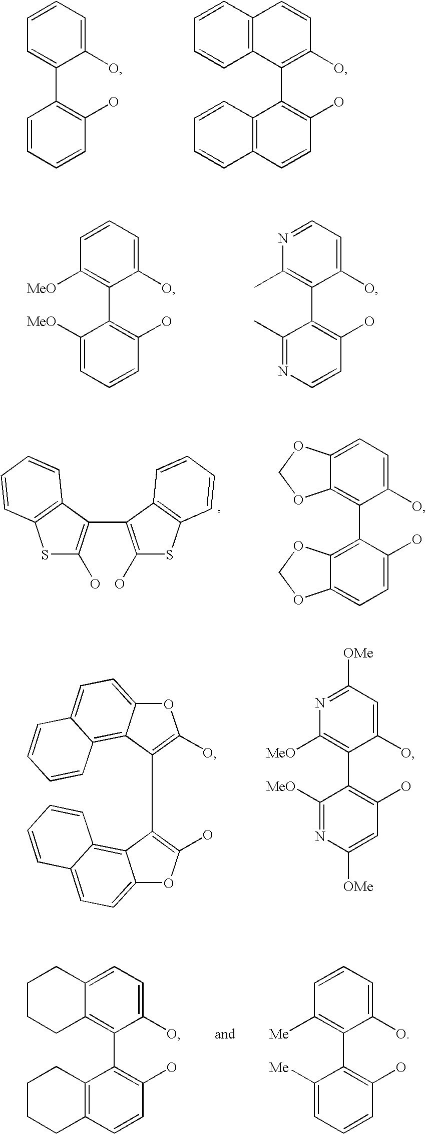

13. A catalytic complex according to the formula:
MSXₙL
Where M is a transition metal which is selected from the group consisting of iridium, tungsten, molydenum, rhodium, ruthenium, nickel, palladium, platinum, copper and silver;
S is a coordinating ligand;
X is a counterion;
n is an integer from 0 to 6; and
L is a phosphoramidite ligand according to claim 1.

14. A catalytic complex according to the formula:
M′SₘXₖL
Where M′ is a transition metal which is preferably selected from the group consisting of iridium, rhodium, ruthenium, copper and silver;
S is a coordinating ligand;
X is a counterion;
m is an integer from 0 to 6;
k is an integer from 0 to 6; and
L is a phosphoramidite ligand according to claim 1.

15. The catalytic complex according to claim 13 wherein said coordinating ligand is selected from the group consisting of ethylene, maleic anhydride, 1,5-cyclooctadiene, cyclooctene, 1,3-butadiene, 2,3-dimethyl-1,3-butadiene, 2,5-norbornadiene, benzene, hexamethyl benzene, cymene, cumene, cyclopentadiene, pentamethylcyclopentadiene, 1,2-diaminoethane, (R,R)-1,2-cyclohexanediamine, (S,S)-1,2-diphenyl-1,2-diaminoethane, (S,S)-1,2-dicyclohexyl-1,2-diaminoethane, (S)-1,1′-bis-(p-methoxyphenyl)-1,2-propanediamine and mixtures thereof

16. The complex according to claim 13 wherein said coordinating ligand is 1,5-cyclooctadiene (COD) or 2,5-norbornadiene.

17. The complex according to claim 13 wherein said counterion is selected from the group consisting of Cl, Br, I, acetate, BF₄, PF₆, ClO₄, p-toluene sulfonate, benzene phosphonate, tetra-pentafluorophenylborate, Li, Na, K, Mg, Ca, ammonium, alkyl-substituted ammonium and mixtures thereof.

18. A method of making a catalyst complex comprising a metal complex of a phosphoramidite, comprising the step of combining a catalyst precursor MSXₙ and a phosphoramidite compound according to claim 13 in a solvent in the presence of an optional base under conditions which form the catalyst complex MSXₙL,
Where M is a transition metal selected from the group consisting of iridium, rhodium, ruthenium, tungsten, molybdenum, nickel, palladium and platinum;
S is a coordinating ligand;
X is a counterion;
n is an integer from 0 to 6 and
L is a phosphoramidite ligand.

19. A method of making a catalyst complex comprising a metal complex of a phosphoramidite, comprising the step of combining a catalyst precursor M′SₘXₖ and a phosphoramidite compound according to claim 13 in a solvent in the presence of an optional base under conditions which form the catalyst complex M′SₘXₖL,
where M′ is a transition metal selected from the group consisting of iridium, rhodium, ruthenium, copper and silver;
S is a coordinating ligand;
X is a counterion;
m is an integer from 0 to 6;
k is an integer from 0 to 6; and
L is a phosphoramidite ligand.

20. The method according to claim 18 wherein said coordinating ligand is selected from the group consisting of ethylene, maleic anhydride, 1,5-cyclooctadiene, cyclooctene, 1,3-butadiene, 2,3-dimethyl-1,3-butadiene, 2,5-norbornadiene, benzene, hexamethyl benzene, cymene, cumene, cyclopentadiene, pentamethylcyclopentadiene, 1,2-diaminoethane, (R,R)-1,2-cyclohexanediamine, (S,S)-1,2-diphenyl-1,2-diaminoethane, (S,S)-1,2-dicyclohexyl-1,2-diaminoethane, (S)-1,1′-bis-(p-methoxyphenyl)-1,2-propanediamine and mixtures thereof

21. The method according to claim 18 wherein said coordinating ligand is 1,5-cyclooctadiene (COD) or 2,5-norbornadiene.

22. The method according to any of claim 18 wherein said counterion is selected from the group consisting of Cl, Br, I, acetate, BF₄, PF₆, ClO₄, p-toluene sulfonate, benzene phosphonate, tetra-pentafluorophenylborate, Li, Na, K, Mg, Ca, ammonium, alkyl-substituted ammonium and mixtures thereof.

23. A method of preparing chemical products from the formation of a carbon-carbon bond between (a) an achiral or racemic allylic ester, allylic carbonate or allylic halide and (b) a carbanion reagent derived from a 1,3 dicarbonyl compound, cyanoester, or carbonyl compound with a P-sulfone or phosphate, or the corresponding neutral reagent that is converted into a carbanionic reagent in the presence of base, the reacting step taking place in a solvent and an optional metal salt or base, optionally at a temperature above or below ambient temperature, and in the presence of a catalyst composition, the catalyst composition comprising:
(1) a catalyst precursor having the structure MSXₙ;
wherein M is a transition metal selected from the group consisting of iridium, rhodium, ruthenium, molybdenum, tungsten, nickel, palladium and platinum;
S is a coordinating ligand;
X is a counterion; and
n is an integer from 0 to 6; and
(2) a phosphoramidite ligand having the structure: 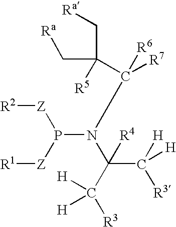
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent or is H, an optionally substituted C₁-C₁₂ alkyl group or an optionally substituted
(CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring, or an aromatic or heteroaromatic ring, R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center;
under conditions that form said chemical product.

24. The method according to claim 23 wherein said carbonanion is obtained from a cyanoacetate, α-cyanoketone, malonate, 1,3-diketone, azlactone or imine-protected α-aminoacid.

25. A method of preparing an allylic ether enantioselectively, the method comprising the steps of reacting (a) an achiral or racemic allylic ester, allylic carbonate or allylic halide and (b) a reactant containing an O—H bond, or a salt thereof, the reacting step taking place in a solvent, optionally at a temperature above or below ambient temperature, in the presence of a catalyst composition, the catalyst composition comprising:
(1) a catalyst precursor having the structure MSXₙ;
wherein M is a transition metal selected from the group consisting of iridium, rhodium, ruthenium, molybdenum, tungsten, nickel, palladium and platinum;
S is a coordinating ligand;
X is a counterion; and
n is an integer from 0 to 6; and
(2) a phosphoramidite ligand having the structure: 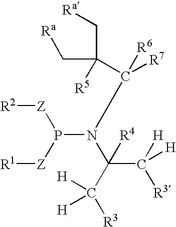
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂),-aromatic group;
R⁵ is absent or is H, an optionally substituted C₁-C₁₂ alkyl group or an optionally substituted
(CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring, or an aromatic or heteroaromatic ring, R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center;
under conditions that form said allylic ether.

26. A method of enantioselectively hydrogenating an olefin compound, the method comprising the steps of reacting (a) hydrogen and (b) an olefin compound, the reacting step taking place in a solvent, optionally at a temperature above or below ambient temperature, in the presence of a catalyst composition, the catalyst composition comprising:
(1) a catalyst precursor having the structure M′SmXk;
wherein M is a transition metal selected from the group consisting of iridium, rhodium and ruthenium;
S is a coordinating ligand;
X is a counterion; and
m is an integer from 0 to 6;
k is an integer from 0 to 6; and
(2) a phosphoramidite ligand having the structure: 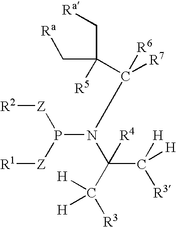
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent or is H, an optionally substituted C₁-C₁₂ alkyl group or an optionally substituted
(CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring, or an aromatic or heteroaromatic ring, R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center under conditions of hydrogenating said olefin; and
isolating a hydrogenated olefin.

27. A method of enatioselectively reacting a compound containing a nucleophilic group with an alpha, beta-unsaturated carbonyl compound or nitroalkene to form a conjugate addition compound, the method comprising the steps of reacting (a) said compound containing a nucleophilic group and (b) said alpha, beta-unsaturated carbonyl compound, the reacting step taking place in a solvent, optionally at a temperature above or below ambient temperature, in the presence of a catalyst composition, the catalyst composition comprising:
1) a catalyst precursor having the structure M′SₘXₖ;
wherein M is a transition metal selected from the group consisting of iridium, rhodium and ruthenium;
S is a coordinating ligand;
X is a counterion; and
m is an integer from 0 to 6;
k is an integer from 0 to 6; and
(2) a phosphoramidite ligand having the structure: 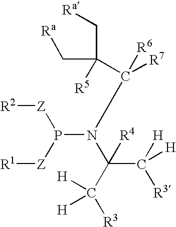
Where Z is a group bound to phosphorous through C, O, N or S, preferably O;
R¹ and R² are independently an optionally substituted C₁-C₁₂ alkyl group, an optionally substituted (CH₂)ₙ-aromatic group or (CH₂)ₙ-heteroaromatic group, or are linked together to form an optionally substituted aliphatic or (CH₂)ₙ-aromatic dianion of a diol, diamine, dithiol, aminoalcohol, aminothiol or alcoholthiol group;
R^{3′} and R³ are each independently H, an optionally substituted C₁-C₁₂ alkyl group or an optionally substituted (CH₂)ₙ-aromatic group with the proviso that R^{3′} and R³ are not both H, or together R³′ and R³ form an optionally substituted C₅-C₁₅ saturated or unsaturated carbocyclic ring;
R⁴ is H, an optionally substituted C₁-C₁₂ alkyl group or an optionally substituted (CH₂)ₙ-aromatic group;
R⁵ is absent or is H, an optionally substituted C₁-C₁₂ alkyl group or an optionally substituted
(CH₂)ₙ-aromatic group;
R^{a} and R^{a′} are each independently H or a C₁-C₃ alkyl group, or R^{a} and R^{a′} together with the carbon to which they are attached form a optionally substituted C₅-C₁₅ saturated or unsaturated carbocyclic or heterocyclic ring, or an aromatic or heteroaromatic ring;
R⁶ and R⁷ are each independently H, an optionally substituted C₁-C₁₂ alkyl group or an optionally substituted (CH₂)ₙ-aromatic group, with the proviso that R⁵, R⁶ and R⁷ cannot simultaneously be H, and when R^{a} and R^{a′}, together with the carbon to which they are attached, form a carbocyclic ring, a heterocyclic ring, or an aromatic or heteroaromatic ring, R⁵ is absent or is preferably H; R⁶ and R⁷ are preferably H or CH₃; and
Each n is independently 0, 1, 2, 3, 4, 5 or 6 and wherein at least one of the carbon atoms attached to the nitrogen of the phosphoramidite group is a chiral center under conditions to form a conjugate addition compound; and
isolating said conjugate addition compound.

28. The method according to claim 23 wherein said phosphoramidite ligand L has the chemical structure: 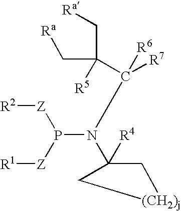
Wherein Z, R¹, R², R⁴, R⁵, R⁶, R⁷, R^{a} and R^{a′} are the same as in claim 23 and j is an integer from 2 to 12.

29. The method according to any of claim 23 wherein R⁵ is absent or is H.

30. The method according to any of claim 23 wherein R⁶ and R⁷ are each independently H or CH₃.

31. The method according to claim 23 wherein said phosphoramidite ligand has the chemical structure: 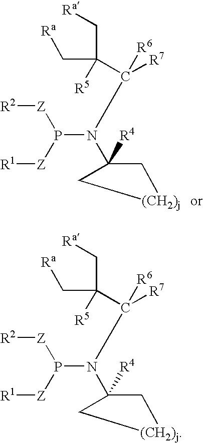
Wherein Z, R¹, R², R⁴, R⁵, R⁶, R⁷ , R^{a} and R^{a′} and j are the same as in claim 23.

32. The method according to any of claim 23 wherein R^{a} and R^{a′}, together with the carbon to which they are attached, form a carbocyclic ring, heterocyclic ring or an aromatic or heteroaromatic ring.

33. The method according to claim 23 wherein the group 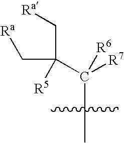
provides a substituted benzyl or naphthylmethyl group connected to the nitrogen atom, such that R⁵ is absent and R⁶ is H and R⁷ is H or CH₃.

34. The method according to claim 23 wherein R^{3′} and R³ together with the carbon to which they are attached from a carbocyclic group and R⁴ is H or a methyl group.

35. The method according to claim 23 wherein R¹ and R² are linked and form a biphenyl or binaphthyl group.

36. The method according to claim 23 wherein the group 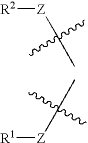
forms an O—Cₖ—O group.

37. The method according to claim 36 wherein said O—Cₖ—O group is an aliphatic or aromatic diolate.

38. The method according to claim 23 wherein said 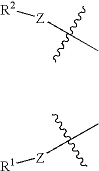
group is selected from the group consisting of:

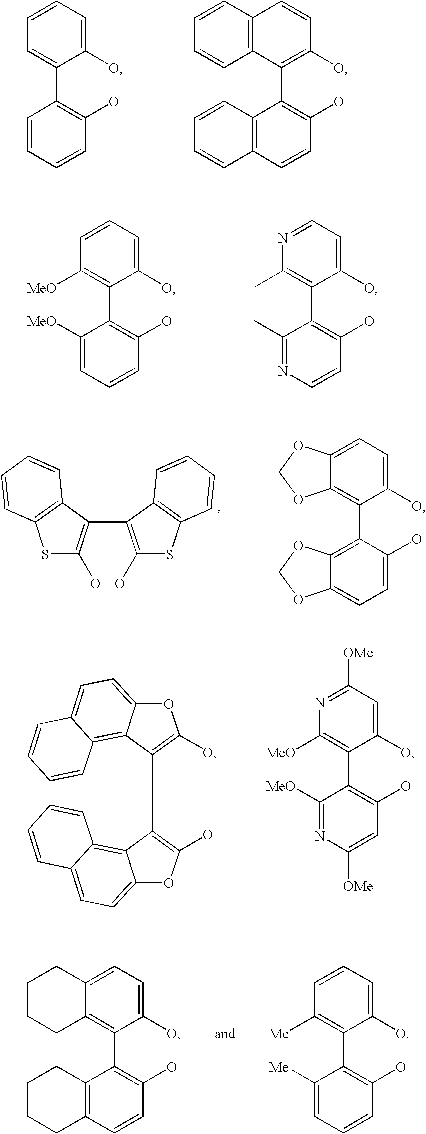

39. The method according to claim 23 wherein said achiral or racemic allylic ester is selected from the group consisting of: Ph-CH═CH—CH₂—OAc, 4-MeO—C₆H₄—CH═CH—CH₂—OAc, 4-NO₂—C₆H₄—CH═CH—CH₂—OAc, 2-MeO—C₆H₄—CH═CH—CH₂—OAc, 2-furyl-CH═CH—CH₂—OAc, n-C₃H₇—CH═CH—CH₂—OAc, Me-CH═CH—CH₂—OAc, n-Pr—CH═CH—CH₂—OAc, i-Pr—CH═CH—CH₂—OAc, CH₃—CH═CH—CH═CH—CH₂—OAc and mixtures thereof.

40. The method according to claim 23 wherein said achiral or racemic allylic carbonate is selected from the group consisting of Ph-CH═CH—CH₂—OCOOMe, 4-MeO—C₆H₄—CH═CH—CH₂—OCOOMe, 4-NO₂—C₆H₄—CH═CH—CH₂—OCOOMe, 2-MeO—C₆H₄—CH═CH—CH₂—OCOOMe, 2-furyl-CH═CH—CH₂—OCOOMe, n-C₃H₇—CH═CH—CH₂—OCOOMe, Me-CH═CH—CH₂—OCOOMe, n-Pr—CH═CH—CH₂—OCOOMe, i-Pr—CH═CH—CH₂—OCOOMe, CH₃—CH═CH—CH═CH—CH₂—OCOOMe and mixtures thereof.

41. The method according to claim 23 wherein said achiral or racemic allylic halide is selected from the group consisting of Ph-CH═CH—CH₂—X, 4-MeO—C₆H₄—CH═CH—CH₂—X, 4-NO₂—C₆H₄—CH═CH—CH₂—X, 2-MeO—C₆H₄—CH═CH—CH₂—X, 2-furyl-CH═CH—CH₂—X, n-C₃H₇—CH═CH—CH₂—X, Me-CH═CH—CH₂—X, n-Pr—CH═CH—CH₂—X, i-Pr—CH═CH—CH₂—X, and CH₃—CH═CH—CH═CH—CH₂—X, wherein X is selected from a halide atom such as F, Cl, Br, and I.
